(19) United States
(12) Patent Application Publication
(10) Pub. No.: US 2016/0220639 A1
(43) Pub. Date: Aug. 4, 2016

(54) METHODS AND COMPOSITIONS FOR TREATING BONE DISEASES

(71) Applicants: NEW YORK UNIVERSITY, New York, NY (US); CORNELL UNIVERSITY, Ithaca, NY (US)

(72) Inventors: Bruce N. Cronstein, New York, NY (US); Aranzazu Mediero Munoz, New York, NY (US); Barbara Hampstead, New York, NY (US)

(21) Appl. No.: 15/021,023

(22) PCT Filed: Sep. 10, 2014
(86) PCT No.: PCT/US14/54906

§ 371 Date: Mar. 10, 2016

Related U.S. Application Data
(60) Provisional application No. 61/876,288, filed on Sep. 11, 2013.

(51) Int. Cl. A61K 38/18; A61K 45/06

(52) U.S. Cl. CPC A61K 38/185; A61K 45/06

(57) ABSTRACT

The invention provides methods and compositions for stimulating or promoting bone generation and regeneration, for stimulating or increasing differentiation, activation or function of osteoblasts, for inhibiting or decreasing differentiation, activation or function of osteoclasts, and for treating bone diseases such as osteonecrosis by administering to a subject a therapeutically effective amount of a pro-nerve growth factor (proNGF) or a pro-brain derived neurotrophic factor (proBDNF) or an analog, homolog, fragment or derivative thereof. The pro-nerve growth factor (proNGF) or a pro-brain derived neurotrophic factor (proBDNF) or an analog, homolog, fragment or derivative thereof may be a mutated or poorly hydrolyzed form of proNGF or proBDNF.

### STATEMENT OF GOVERNMENT RIGHTS

The present invention was developed, at least in part, using government support under NIH ROI AR054897, ROI NS030687, and PO1-HL046403 awarded by the National Institutes of Health. Therefore, the Federal Government may have certain rights in the invention.

### FIELD OF THE INVENTION

The present invention relates to methods and compositions for stimulating or promoting bone regeneration, bone growth, osteoblast differentiation, activation, and function, for inhibiting or decreasing osteoclast differentiation, activation and function, and for treating, preventing or ameliorating bone disease.

### BACKGROUND OF THE INVENTION

### Osteoblasts

Osteoblasts are mononucleate cells that are responsible for bone formation. They are specialized fibroblasts that in addition to fibroblastic products, express bone sialoprotein and osteocalcin. Osteoblasts produce a matrix of osteoid, which is composed mainly of Type I collagen. Osteoblasts are also responsible for mineralization of this matrix. Calcium, zinc, copper and sodium are some of the minerals required in this process. Bone is a dynamic tissue that is constantly being reshaped by osteoblasts, which are in charge of production of matrix and mineral, and osteoclasts, which break down the tissue. The number of osteoblasts tends to decrease with age, affecting the balance of formation and resorption in the bone tissue, and potentially leading to osteoporosis.

Osteoblasts arise from osteoprogenitor cells located in the deeper layer of periosteum and the bone marrow. Osteoprogenitors are immature progenitor cells that express the master regulatory transcription factor Cbfa1/Runx2. Osteoprogenitors are induced to differentiate under the influence of growth factors, in particular the bone morphogenetic proteins (BMPs). Aside from BMPs, other growth factors including fibroblast growth factor (FGF), platelet-derived growth factor (PDGF) and transforming growth factor beta (TGF-β) may promote the division of osteoprogenitors and potentially increase osteogenesis. Once osteoprogenitors start to differentiate into osteoblasts, they begin to express a range of genetic markers including Osterix, Col1, BSP, M-CSF, ALP, osteocalcin, osteopontin, and osteonectin. Although the term osteoblast implies an immature cell type, osteoblasts are in fact the mature bone cells entirely responsible for generating bone tissue in animals and humans.

### Osteoclasts

Osteoclasts are the bone cells that remove bone tissue by removing its mineralized matrix and breaking up the organic bone (organic dry weight is 90% collagen). This process is known as bone resorption. Osteoclasts are formed by the fusion of cells of the monocyte-macrophage cell line. Osteoclasts are characterized by high expression of tartrate resistant acid phosphatase (TRAP) and cathepsin K. Osteoclasts are found in pits in the bone surface which are called resorption bays, or Howship's Lacunae. Osteoclasts are characterized by a cytoplasm with a homogeneous, “foamy” appearance due to a high concentration of vesicles and vacuoles. These vacuoles are lysosomes filled with acid phophatase. Osteoclast rough endoplasmic reticulum is sparse, and the Golgi complex is extensive. At a site of active bone resorption, the osteoclast forms a specialized cell membrane, the “ruffled border,” that touches the surface of the bone tissue. The ruffled border, which facilitates removal of the bony matrix, is a morphologic characteristic of an osteoclast that is actively resorbing bone. The ruffled border increases surface area interface for bone resorption. The mineral portion of the matrix (called hydroxyapatite) includes calcium and phosphate ions. These ions are absorbed into small vesicles (see endocytosis), which move across the cell and eventually are released into the extracellular fluid, thus increasing levels of the ions in the blood.

Osteoclast formation requires the presence of RANKL (receptor activator of nuclear factor κβ0 ligand) and M-CSF (Macrophage colony-stimulating factor). These membrane bound proteins are produced by neighbouring stromal cells and osteoblasts, thus requiring direct contact between these cells and osteoclast precursors. M-CSF acts through its receptor on the osteoclast, c-fms (colony-stimulating factor 1 receptor), a transmembrane tyrosine kinase-receptor, leading to secondary messenger activation of tyrosine kinase Src. Both of these molecules are necessary for osteoclastogenesis and are widely involved in the differentiation of monocyte/macrophage derived cells. RANKL is a member of the tumor necrosis family (TNF), and is essential in osteoclastogenesis. RANKL knockout mice exhibit a phenotype of osteopetrosis and defects of tooth eruption, along with an absence or deficiency of osteoclasts. RANKL activates NF-κβ (nuclear factor-κβ) and NFATc1 (nuclear factor of activated t cells, cytoplasmic, calcineurin-dependent 1) through RANK. NF-κβ activation is stimulated almost immediately after RANKL-RANK interaction occurs, and is not upregulated. NFATc1 stimulation, however, begins ˜24-48 hours after binding occurs and its expression has been shown to be RANKL dependent. Osteoclast differentiation is inhibited by osteoprotegerin (OPG), which is produced by osteoblasts and binds to RANKL thereby preventing interaction with RANK.

Once activated, osteoclasts move to areas of microfracture in the bone by chemotaxis. Osteoclasts lie in a small cavity called Howship's lacunae, formed from the digestion of the underlying bone. The sealing zone is the attachment of the osteoclast's plasma membrane to the underlying bone. Sealing zones are bounded by belts of specialized adhesion structures called podosomes. Attachment to the bone matrix is facilitated by integrin receptors, such as αvβ3, via the specific amino acid motif Arg-Gly-Asp in bone matrix proteins, such as osteopontin. The osteoclast releases hydrogen ions through the action of carbonic anhydrase (H₂O+CO₂→HCO₃⁻+H⁺) through the ruffled border into the resorptive cavity, acidifying and aiding dissolution of the mineralized bone matrix into Ca²⁺, H₃PO₄, H₂CO₃, water and other substances. Dysfunction of the carbonic anhydrase has been documented to cause some forms of osteopetrosis. Hydrogen ions are pumped against a high concentration gradient by proton pumps, specifically a unique vacuolar-ATPase. This enzyme has been targeted in the prevention of osteoporosis. In addition, several hydrolytic enzymes, such as members of the cathepsin and matrix metalloprotease (MMP) groups, are released to digest the organic components of the matrix. These enzymes are released into the compartment by lysosomes. Of these hydrolytic enzymes, cathepsin K is of most importance.

Osteoclasts are regulated by several hormones, including parathyroid hormone (PTH) from the parathyroid gland, calcitonin from the thyroid gland, and growth factor interleukin 6 (IL-6). This last hormone, IL-6, is one of the factors in the disease osteoporosis, which is an imbalance between bone resorption and bone formation. Osteoclast activity is also mediated by the interaction of two molecules produced by osteoblasts, namely osteoprotegerin and RANK ligand. Note that these molecules also regulate differentiation of the osteoclast.

### Nerve Growth Factor (NGF)

Nerve growth factor (NGF) is a small secreted protein that is important for the growth, maintenance, and survival of certain target neurons (nerve cells). It also functions as a signaling molecule. It is perhaps the prototypical growth factor, in that it is one of the first to be described. Other members of the neurotrophin family that are well recognized include Brain-Derived Neurotrophic Factor (BDNF), Neurotrophin-3 (NT-3), and Neurotrophin 4/5 (NT-4/5). NGF is critical for the survival and maintenance of sympathetic and sensory neurons, and it causes axonal growth, axonal branching and elongation. NGF binds with at least two classes of receptors: the p75 LNGFR (for “low-affinity nerve growth factor receptor”) neurotrophin receptor (p75(NTR)) and TrkA, a transmembrane tyrosine kinase. Both can be dysregulated in human neurodegenerative disorders. NGF binds to receptor tyrosine kinase. TrkA dimerizes and autophosphorylates its tyrosine kinase segment, which leads to the activation of PI 3-kinase, Ras, and PLC-gamma signaling pathways. Alternatively, the p75NTR receptor can form a heterodimer with TrkA which has higher affinity and specificity for NGF. There is evidence that NGF circulates throughout the entire body and is important for maintaining neuronal homeostasis.

Binding interaction between NGF and the TrkA receptor facilitates receptor dimerization and tyrosine residue phosphorylation of the cytoplasmic tail by adjacent Trk receptors. Trk receptor phosphorylation sites operate as Shc adaptor protein docking sites, which undergo phosphorylation by the TrkA receptor. Once the cytoplasmic adaptor protein (Shc) is phosphorylated by the receptor cytoplasmic tail, cell survival is initiated through several intracellular pathways. One major pathway leads to the activation of the serine/threonine kinase, Akt. This pathway begins with the Trk receptor complex-recruitment of a second adaptor protein called growth factor-receptor bound protein-2 (Grb2) along with a docking protein called Grb2-associated Binder-1 (GAB1). Subsequently, phosphatidylinositol-3 kinase (PI3K) is activated, resulting in Akt kinase activation. Study results have shown that blocking PI3K or Akt activity results in death of sympathetic neurons in culture, regardless of NGF presence. However if either kinase is constitutionally active, neurons survive even without NGF. A second pathway contributing to cell survival occurs through activation of the mitogen-activated protein kinase (MAPK) kinase. In this pathway, recruitment of a guanine nucleotide exchange factor by the adaptor and docking proteins leads to activation of a membrane-associated G-protein known as Ras. The guanine nucleotide exchange factor mediates Ras activation through the GDP-GTP exchange process. The active Ras protein phosphorylates several proteins, along with the serine/threonine kinase, Raf Raf, in turn activates the MAPK cascade to facilitate ribosomal s6 kinase (RSK) activation and transcriptional regulation. Both Akt and RSK, components of the PI3K-Akt and MAPK pathways respectively, act to phosphorylate the cyclic AMP response element binding protein (CREB) transcription factor. Phosphorylated CREB translocates into the nucleus and mediates increased expression of anti-apoptotic proteins, thus promoting NGF-mediated cell survival. However, in the absence of NGF, the expression of pro-apoptotic proteins is increased when the activation of cell death-promoting transcription factors such as c-Jun are not suppressed by the aforementioned NGF-mediated cell survival pathways.

There is also evidence that shows that the precursor to NGF, proNGF, may also play important roles, including apoptosis and the acute induction of cytoskeletal reorganization. ProNGF is the uncleaved, precursor protein form of NGF. It consists of the prodomain and the mature domain. Pro NGF is characterized by the cysteine knot motif consisting of three cysteine bridges within the mature domain. The proNGF protein exists as a non-covalently linked homodimer. Pro-Nerve Growth Factor Human Recombinant produced in E. Coli is a non-glycosylated, polypeptide chain containing 241 amino acids. It is commercially available.

The ProNGF precursor is biologically active. ProNGF binds to the 75 kD neurotrophin NTR (a tumor necrosis family member) with a coreceptor, sortilin. High affinity binding between ProNGF, sortilin, and p75NTR can result in programmed cell death (PCD). Study results indicate that superior cervical ganglia neurons that express both p75NTR and TrkA die when treated with proNGF, where as NGF treatment of these same neurons results in survival and axonal growth. Survival and PCD mechanisms are mediated through adaptor protein binding to the death domain of the p75NTR cytoplasmic tail. Survival may occur when recruited cytoplasmic adaptor proteins facilitate signal transduction through tumor necrosis factor receptor members such as TRAF6, which results in the release of nuclear factor κB (NF-κB) transcription activator. NF-κB may regulate nuclear gene transcription to promote cell survival. Alternatively, PCD occurs when TRAF6 and neurotrophin receptor interacting factor (NRIF) are both recruited to activate c-Jun N-terminal kinase (JNK); which phosphorylates c-Jun. The activated transcription factor c-Jun regulates nuclear transcription to increase pro-apoptotic gene transcription.

### Brain-Derived Neurotrophic Factor (BDNF)

Brain-derived neurotrophic factor, also known as BDNF, is a secreted protein that, in humans, is encoded by the BDNF gene. BDNF is a member of the “neurotrophin” family of growth factors, which are related to the Nerve Growth Factor. BDNF acts on certain neurons of the central nervous system and the peripheral nervous system, helping to support the survival of existing neurons, and encourage the growth and differentiation of new neurons and synapses. In the brain, it is active in the hippocampus, cortex, and basal forebrain—areas vital to learning, memory, and higher thinking. BDNF itself is important for long-term memory.

BDNF binds to two receptors on the surface of cells that are capable of responding to this growth factor, TrkB (pronounced “Track B”) and the LNGFR (for low-affinity nerve growth factor receptor, also known as p75). It may also modulate the activity of various neurotransmitter receptors, including the α₇ nicotonic receptor. TrkB is a receptor tyrosine kinase (meaning it mediates its actions by causing the addition of phosphate molecules on certain tyrosines in the cell, activating cellular signaling). There are other related Trk receptors, TrkA and TrkC. Also, there are other neurotrophic factors structurally related to BDNF: NGF, NT-3 (for Neurotrophin-3) and NT-4 (for Neurotrophin-4). TrkB is the primary receptor for BDNF and NT-4, and TrkC is the primary receptor for NT-3. NT-3 binds to TrkA and TrkB as well, but with less affinity (thus the caveat “primary receptor”). The other BDNF receptor, the p75, plays a somewhat less clear role. The p75NTR may signal a cell to die via apoptosis; so, therefore, cells expressing the p75NTR in the absence of Trk receptors may die rather than live in the presence of a neurotrophin.

BDNF is made in the endoplasmic reticulum and secreted from dense-core vesicles. It binds carboxypeptidase E (CPE) and sortilin, and the disruption of this binding has been proposed to cause the loss of sorting of BDNF into dense-core vesicles. The phenotype for BDNF knockout mice is severe with postnatal lethality. Other traits include sensory neuron losses that affect coordination, balance, hearing, taste, and breathing. Knockout mice also exhibit cerebellar abnormalities and an increase in the number of sympathetic neurons. Exercise has been shown to increase the secretion of BDNF at the mRNA and protein levels in the rodent hippocampus, suggesting the potential increase of this neurotrophin after exercise in humans. Caffeine improves recognition memory, and this effect may be related to an increase of the BDNF and TrkB immunocontent in the hippocampus.

BDNF activity is correlated with increased long term potentiation and neurogenesis, which can be induced by physical activity. Long term potentiation is shown to improve learning and memory by strengthening the communication between specific neurons. This was shown in the Morris water maze task in which the role of BDNF was tested in mice. One group of mice exercised on a running wheel while the control group of mice trained under standard conditions lacking physical exercise. When the groups of mice performed the Morris water maze task, the running group significantly increased their learning and memory by decreasing the latency in finding the platform. Bromodeoxyuridine was injected into the mice to label dividing cells which proved to show that the physical exercise enhanced neurogenesis in the dentate gyrus of the hippocampus of the running mice, thus enhancing long term potentiation and memory.

The increase in neurogenesis is hypothesized to increase learning in the mice. MRI scans have shown that exercising mice have a selective increase in cerebral blood flow to the dentate gyrus of the hippocampus, an area of the brain particular to memory and learning, while there was no significant increase observed in other areas of the brain. The control mice group with no exercise did not have the same increase in the hippocampal region. This supporting evidence concludes that exercise selectively increases neurogenesis in the dentate gyrus of the hippocampus.

The mechanism for this is due to BDNF activating the signal transduction cascades, MAP kinase and CAMKII, which regulate the expression of the transcription factor, CREB, and protein synapsin I. The mitochondria and the uncoupling protein, UCP2, which is mainly present in the brain's mitochondria, have been thought to interact with this signal transduction cascade during physical activity. CREB and synapsin I both play a role in enhancing plasticity by changing the structure of the neuron and strengthening its signaling capability, therefore affecting long term potentiation. CREB specifically aids in spatial learning and regulating gene expression, while synapsin I modulates the release of neurotransmitters and affects the actin cytoskeleton of the cell which enhances the signaling capability of the neuron by changing its shape and density.

A recombinant human precursor form of Brain-Derived Neurotrophic Factor/proBDNF is produced in E. coli is a non-glycosylated polypeptide chain containing 247 amino acids and forms a homodimer. It is commercially available.

### Interaction Between Peripheral Nerves and Bone

A peculiar form of destructive arthritis (now known as Charcot joint or neuropathic arthropathy) was reported in the 1860's in a patient with advanced syphilis affecting the central nervous system. Subsequently, the phenomenon was described in patients with other types of medical problems affecting the peripheral nerves. Because of the development of penicillin for the treatment of syphilis, the most common cause for neuropathic arthropathy in the United States today is Diabetes Mellitus with its associated peripheral neuropathy although many different types of peripheral neuropathy can lead to the development of neuropathic arthropathy. The main consequence of neuropathic arthritis is deformity of the joint (usually the foot and ankle) leading to progressive difficulty in walking and other activities. At present there is very little solid information regarding the process by which loss of sensation (due to loss of innervation) leads to joint problems although the most commonly cited hypothesis is that there is damage to the joint resulting from numerous small traumas that the patient does not feel. Despite observations made over the past 150 years there is little evidence to support this theory. The contribution of peripheral nerves to maintaining bone and the interaction between bone and peripheral nerves has not been well explored.

Recent experiments in patients suggest a novel interaction between peripheral nerves and bone. One experimental approach to the treatment of osteoarthritis, a common painful condition of the joints, is the administration of antibodies to Nerve Growth Factor (NGF) to provide symptomatic relief by preventing the regeneration and maintenance of pain fibers (pain sensing nerves). While effective for the symptomatic treatment of osteoarthritis, one of the severe toxic side effects has been osteonecrosis or destruction of bone, often in the hip or other weight-bearing joints (aside from the bones of the joint being treated). Although the trials of anti-NGF continue despite this toxicity little is known about how osteonecrosis develops when local NGF levels are reduced.

NGF is reduced in diabetics and early attempts to treat peripheral neuropathy in diabetes with NGF provided some clinical improvement but toxicities of the agent led to the abandonment of this form of therapy for peripheral neuropathy in diabetics a number of years ago. NGF is secreted by cells as a large precursor protein (proNGF) which is cleaved by enzymes outside of the cell into an active form. It is now appreciated that, like the mature form of NGF, proNGF can activate intracellular signaling by binding its own receptors on neurons and other cells (p75, SORCS2 and sortilin). It should be noted that antibodies to NGF will invariably bind to proNGF as well so that anti-NGF antibodies used in patients will also deplete local proNGF as well as NGF.

Diabetic neuropathy and other types of peripheral neuropathies are very common problems that are increasing in magnitude. Understanding how loss of function of peripheral nerves, whether as the result of an injury, diabetes or an infection, can lead to destructive arthritis is critical for designing new treatments to ameliorate or even prevent the joint destruction associated with neuropathy.

All publications, patent applications, patents and other reference material mentioned are incorporated by reference in their entirety. In addition, the materials, methods and examples are only illustrative and are not intended to be limiting. The citation of references herein is not to be construed as an admission that the references are prior art to the present invention.

### SUMMARY OF THE INVENTION

The invention provides novel methods for stimulating or promoting bone growth, bone density or bone regeneration, methods for treating bone diseases, methods for stimulating, activating or increasing the function of osteoblasts, and methods for inhibiting or decreasing the function of osteoclasts, and agents and compositions effective for the same. The methods may be generally practiced in vivo, ex vivo, or in vitro.

In a first aspect, the invention provides a method for stimulating or promoting bone growth, bone density or bone regeneration by administering to a subject a therapeutically effective amount of a pro-nerve growth factor (proNGF) or a pro-brain derived neurotrophic factor (proBDNF) or an analog, homolog, fragment or derivative thereof.

The administering is preferably performed in vivo, though the methods encompass in vitro administration. The administering may be performed once, twice, three, four, five, six or seven or more times daily, weekly, monthly, quarterly or annually, and the administering may be performed intravenously, subcutaneously or even orally.

The pro-nerve growth factor (proNGF) or a pro-brain derived neurotrophic factor (proBDNF) or an analog, homolog, fragment or derivative thereof may be a mutated or poorly hydrolyzable form of proNGF or proBDNF. The pro-nerve growth factor (proNGF) or a pro-brain derived neurotrophic factor (proBDNF) or an analog, homolog, fragment or derivative thereof may stimulate osteoblast differentiation, activation or function or inhibit osteoclast differentiation, activation or function.

In a second aspect, the invention provides a method for stimulating differentiation or activation of osteoblasts or increasing function of osteoblasts comprising administering to a subject a therapeutically effective amount of a pro-nerve growth factor (proNGF) or a pro-brain derived neurotrophic factor (proBDNF) or an analog, homolog, fragment or derivative thereof.

The administering is preferably performed in vivo, though the methods encompass in vitro administration. The administering may be performed once, twice, three, four, five, six or seven or more times daily, weekly, monthly, quarterly or annually, and the administering may be performed intravenously, subcutaneously or even orally.

The pro-nerve growth factor (proNGF) or a pro-brain derived neurotrophic factor (proBDNF) or an analog, homolog, fragment or derivative thereof may be a mutated or poorly hydrolyzed form of proNGF or proBDNF. The pro-nerve growth factor (proNGF) or a pro-brain derived neurotrophic factor (proBDNF) or an analog, homolog, fragment or derivative thereof may stimulate osteoblast differentiation, activation or function or inhibit osteoclast differentiation, activation or function.

In a third aspect, the invention provides a method for inhibiting differentiation or activation of osteoclasts or decreasing function of osteoclasts comprising administering to a subject a therapeutically effective amount of a pro-nerve growth factor (proNGF) or a pro-brain derived neurotrophic factor (proBDNF) or an analog, homolog, fragment or derivative thereof.

The administering is preferably performed in vivo, though the methods encompass in vitro administration. The administering may be performed once, twice, three, four, five, six or seven or more times daily, weekly, monthly, quarterly or annually, and the administering may be performed intravenously, subcutaneously or even orally.

The pro-nerve growth factor (proNGF) or a pro-brain derived neurotrophic factor (proBDNF) or an analog, homolog, fragment or derivative thereof may be a mutated or poorly hydrolyzed form of proNGF or proBDNF. The pro-nerve growth factor (proNGF) or a pro-brain derived neurotrophic factor (proBDNF) or an analog, homolog, fragment or derivative thereof may stimulate osteoblast differentiation, activation or function or inhibit osteoclast differentiation, activation or function.

In a fourth aspect, the invention provides methods for treating, ameliorating or preventing a bone disease or a condition in a subject having such a disease or condition, or in a subject at risk for developing such a disease or condition. The methods feature administering to the subject a therapeutically effective amount of a pro-nerve growth factor (proNGF) or a pro-brain derived neurotrophic factor (proBDNF) or an analog, homolog, fragment or derivative thereof.

The administering is preferably performed in vivo, though the methods encompass in vitro administration. The administering may be performed once, twice, three, four, five, six or seven or more times daily, weekly, monthly, quarterly or annually, and the administering may be performed intravenously, subcutaneously or even orally.

The methods may be particularly useful when it is desired to stimulate or increase osteoblast function, differentiation, or activation or to inhibit or decrease osteoclast function, differentiation or activation. The pro-nerve growth factor (proNGF) or a pro-brain derived neurotrophic factor (proBDNF) or an analog, homolog, fragment or derivative thereof may be a mutated or poorly hydrolyzed form of proNGF or proBDNF. The pro-nerve growth factor (proNGF) or a pro-brain derived neurotrophic factor (proBDNF) or an analog, homolog, fragment or derivative thereof may stimulate osteoblast differentiation, activation or function or inhibit osteoclast differentiation, activation or function.

The disease or condition may be, for instance, osteoporosis, juvenile osteoporosis, bone loss due to or associated with the onset of menopause, osteoporotic fractures, giant cell tumors of bone, renal osteodystrophy, osteogenesis imperfecta, hypercalcemia, hyperparathyroidism, osteomalacia, osteohalisteresis, osteolytic bone disease, osteonecrosis, Paget's disease of bone, bone loss due to rheumatoid arthritis, inflammatory arthritis, osteomyelitis, corticosteroid treatment, metastatic bone diseases or malignancy-induced osteoporosis and bone lysis, childhood idiopathic bone loss, periodontal bone loss, age-related loss of bone mass, osteotomy and bone loss associated with prosthetic ingrowth, other forms of osteopenia, and in other conditions where facilitation of bone repair or replacement is desired such as bone fractures, bone defects, plastic surgery, dental and other implantations. In some particular embodiments, the bone disease may be one or more of avascular necrosis, neuropathic arthropathy (Charcot Joint), osteoporosis, metastatic lesions, etc. In addition the present invention provides non-hydrolyzed, mutated, etc proNGF or proBDNF to promote bone growth, bone generation, regeneration and fracture healing.

In some instances, bone generation or regeneration or differentiation or activation of osteoblasts may be increased by 10%, 20%, 30%, 40%, 50%, 60%, 70%, 80%, 90%, 100%, or by two fold, three fold, four fold, five fold, ten fold or more relative to normal. Likewise, in some instances, the speed of bone generation or regeneration or number of differentiated or stimulated osteoblasts may be increased by 10%, 20%, 30%, 40%, 50%, 60%, 70%, 80%, 90%, 100%, or by two fold, three fold, four fold, five fold, ten fold or more relative to normal. Likewise, in some instances, the number of differentiated or stimulated osteoclasts may be decreased by 10%, 20%, 30%, 40%, 50%, 60%, 70%, 80%, 90%, 100%, or by two fold, three fold, four fold, five fold, ten fold or more relative to normal.

In some particular embodiments, a therapeutically effective amount of a pro-nerve growth factor (proNGF) or a pro-brain derived neurotrophic factor (proBDNF) or an analog, homolog, fragment or derivative thereof may be used in combination with one or more drugs useful in inhibiting bone resorption or inhibiting differentiation or stimulation of osteoclasts or in stimulating or promoting bone generation or regeneration or stimulation or activation or function of osteoblasts or a combination of any of these agents.

The a therapeutically effective amount of a pro-nerve growth factor (proNGF) or a pro-brain derived neurotrophic factor (proBDNF) or an analog, homolog, fragment or derivative thereof may be administered alone or in combination with one or more other compounds or agents for inhibiting bone resorption or osteoclast differentiation or for stimulating or increasing bone regeneration or bone growth or stimulating osteoblast differentiation. Such other compounds may be, for instance, anti-inflammatory compounds, bisphosphonates or growth factors or an adenosine receptor agonist or antagonist. The a therapeutically effective amount of a pro-nerve growth factor (proNGF) or a pro-brain derived neurotrophic factor (proBDNF) or an analog, homolog, fragment or derivative thereof may be administered or provided in a matrix such as, for example a calcium sulfate matrix, a calcium phosphate matrix or bovine collagen. Such a matrix may be directly applied to bone.

In a fifth aspect, the present invention provides a pharmaceutical composition comprising a therapeutically effective amount of a pro-nerve growth factor (proNGF) or a pro-brain derived neurotrophic factor (proBDNF) or an analog, homolog, fragment or derivative thereof alone or in combination with one or more compounds or agents effective for inhibiting bone resorption, for inhibiting osteoclast differentiation, activation or function or for promoting osteoblast differentiation, activation or function. The therapeutically effective amount of a pro-nerve growth factor (proNGF) or a pro-brain derived neurotrophic factor (proBDNF) or an analog, homolog, fragment or derivative thereof may be formulated and administered alone or together. The pharmaceutical composition(s) comprising the therapeutically effective amount of a pro-nerve growth factor (proNGF) or a pro-brain derived neurotrophic factor (proBDNF) or an analog, homolog, fragment or derivative thereof and the one or more compounds or agents may be administered concurrently or sequentially. In another particular embodiment, the one or more compounds or agents effective for inhibiting bone resorption or osteoclast differentiation and stimulation or for stimulating osteoblast differentiation and activation may be selected from the group consisting of those effective for stimulating bone density and those effective for inhibiting or reducing inflammation. The pharmaceutical compositions may be delivered intravenously, subcutaneously, orally or parenterally. They may be delivered as an immediate release formulation or as a slow or sustained release formulation. In some particular embodiments, the compositions are delivered on the surface of a prosthetic device or are delivered in the very matrix of a prosthetic device.

In another more particular embodiment, the pharmaceutical composition may also contain one or more drugs selected from among anti-inflammatory agents, growth factors, bone morphogenetic protein, and soluble RANK. In some instances, the pharmaceutical composition may be administered or provided in a matrix such as, for example a calcium sulfate matrix. Such a matrix may be directly applied to bone defects to promote bone formation.

Other objects and advantages will become apparent to those skilled in the art from a review of the following description which proceeds with reference to the following illustrative drawings.

### BRIEF DESCRIPTION OF THE FIGURES

FIG. 1 represents in vitro and in vivo characterization of bones. A) WT, proNGF/+, proBDNF/+, BDNF+/− and TrkB+/− mice (n=5 each) osteoclast primary culture cells were fixed and stained for TRAP after being cultured for 7 days in the presence of M-CSF/RANKL. TRAP-positive cells containing three or more nuclei were counted as osteoclasts. B) WT, proNGF/+, proBDNF/+, BDNF+/− and TrkB+/− mice (n=5 each) osteoblast primary culture cells were fixed and stained for Alizarin Red after being cultured for 10 days in the presence of osteogenic media (μMEM containing 1 μM dexamethasone, 50 μg/ml ascorbic acid, and 10 mM β-glycerophosphate). Alizarin Red staining intensity was quantified using SigmaScan software. C) Representative high resolution microCT images. 3D images reconstruction of the femurs revealed increased bone mass in proNGF/+, proBDNF/+, BDNF+/− and TrkB+/− mice (n=3 each) compared with their WT littermates (n=3 each). D) Digital morphometric analysis of microCT images from WT and proNGF/+, proBDNF/+, BDNF+/− and TrkB+/− mice. All data are expressed as means±SEM. ***p<0.001, **p<0.01, *p<0.5 related to WT (ANOVA).

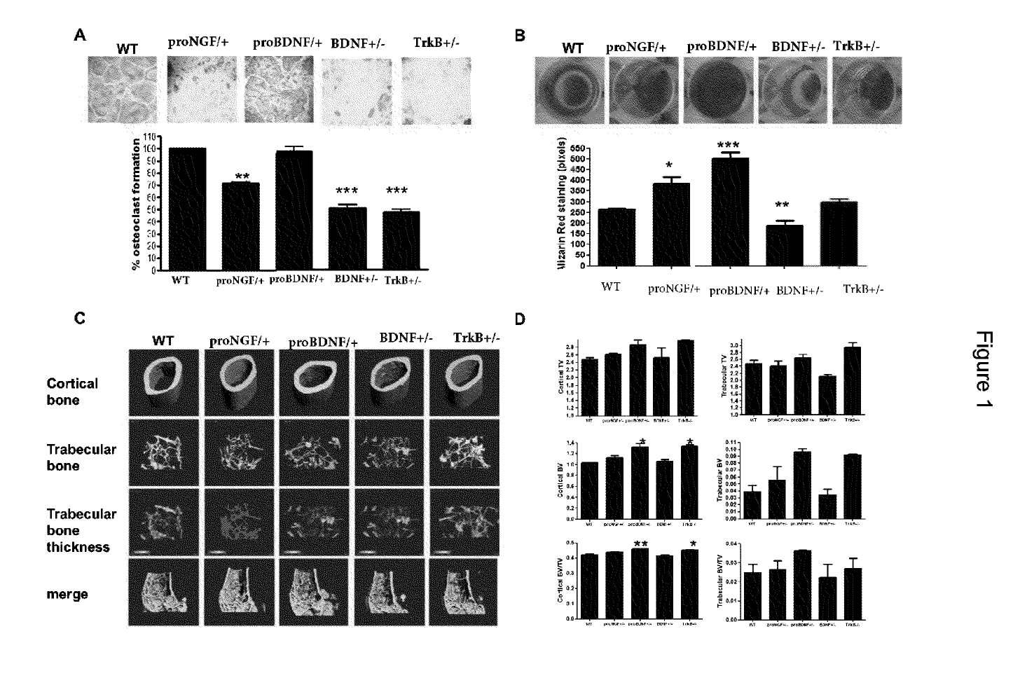

FIG. 2 represents expression of osteoclast differentiation marker mRNA. Changes in Cathepsin K, NFATc1 and Ostepontin mRNA in M-CSF/RANKL precursors after seven days of osteoclast differentiation in WT and proNGF/+, proBDNF/+, BDNF+/− and TrkB+/− mice (n=2 each).

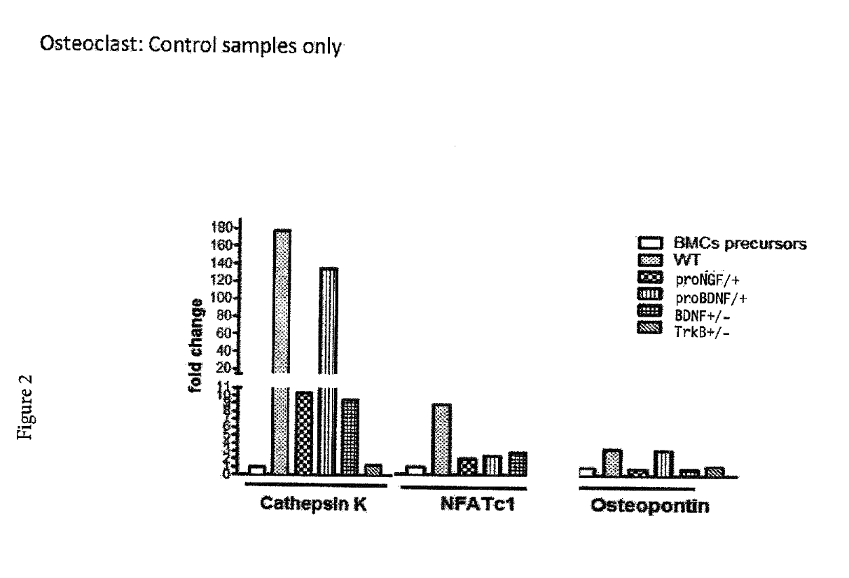

FIG. 3 represents expression of RANKL and Osteoprotegerin mRNA. Changes in RANKL and steoprotegerin (OPG) mRNA in osteogenic precursors after the ten days of osteoblast differentiation in WT, proBDNF/+, and BDNF+/− mice (n=2 each).

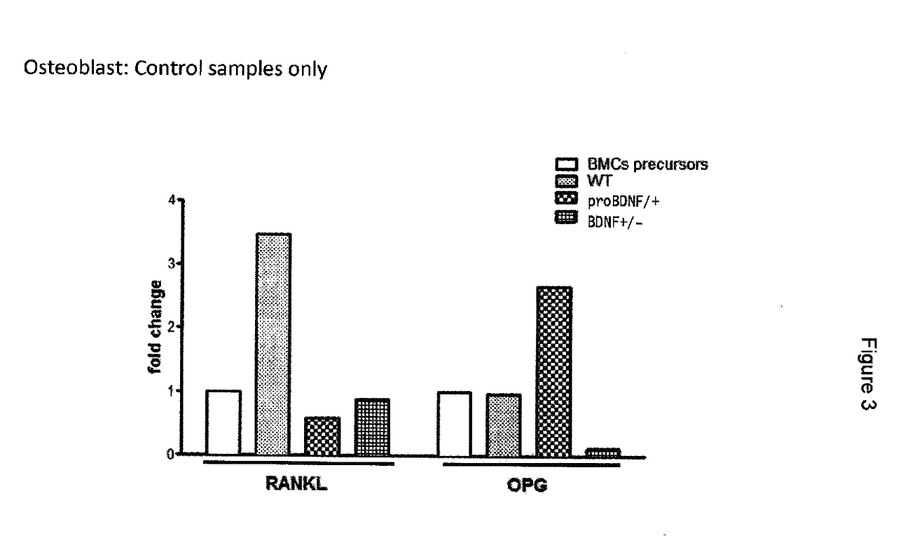

FIG. 4 represents protein and messenger expression for proNGF receptors. A) p75 and sortilin expression were analyzed 24 hours after RANKL stimulation in WT mice M-CSF/RANKL precursors (n=4). p75 mRNA expression was analyzed 24 hours after RANKL stimulation in WT mice M-CSF/RANKL precursors (n=4). B) p75 and sortilin expression were analyzed 24 hours after Osteogenic media (αMEM containing 1 μM dexamethasone, 50 μg/ml ascorbic acid, and 10 mM β-glycerophosphate) stimulation in WT mice M-CSF/RANKL precursors (n=4). p75 mRNA expression was analyzed 24 hours after RANKL stimulation in WT mice osteogenic media precursors (n=4). All data are expressed as mean±SEM. ***p<0.001, **p<0.01 vs. control (ANOVA).

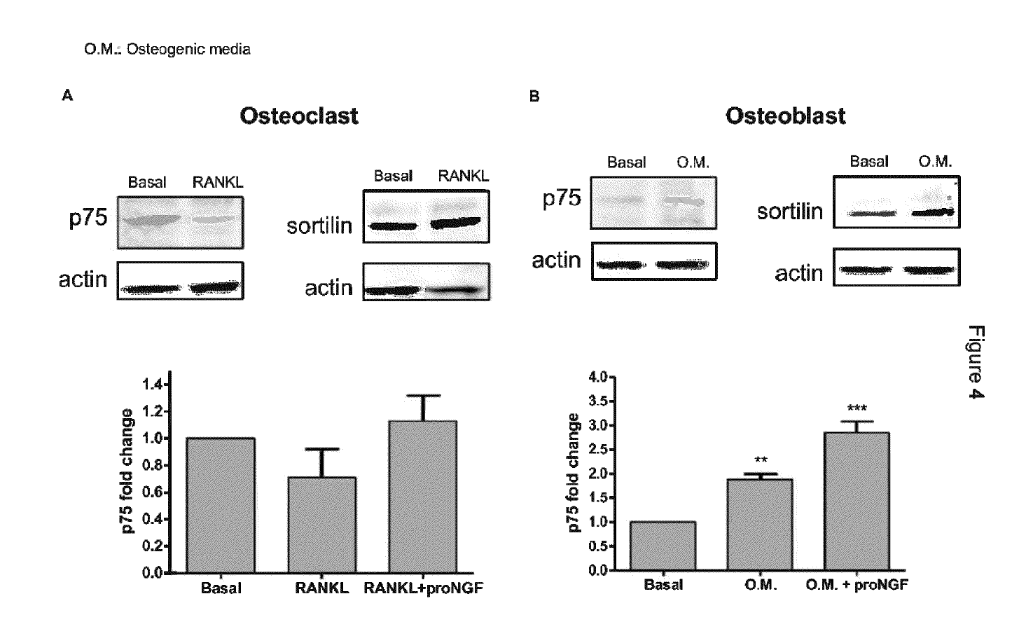

FIG. 5 demonstrates that proNGF modulates osteoclast and osteoblast differentiation. A) WT mice (n=4 each) osteoclast primary culture cells were fixed and stained for TRAP after being cultured for 7 days in the presence of M-CSF/RANKL alone or with recombinant proNGF (20 ng/ml). TRAP-positive cells containing three or more nuclei were counted as osteoclasts. B) WT mice (n=4 each) osteoblast primary culture cells were fixed and stained for Alizarin Red after being cultured for 10 days in the presence of osteogenic media (αMEM containing 1 μM dexamethasone, 50 μg/ml ascorbic acid, and 10 mM β-glycerophosphate). Alizarin Red staining intensity was quantified using SigmaScan software. All data are expressed as mean±SEM. ***p<0.001 vs. control (ANOVA).

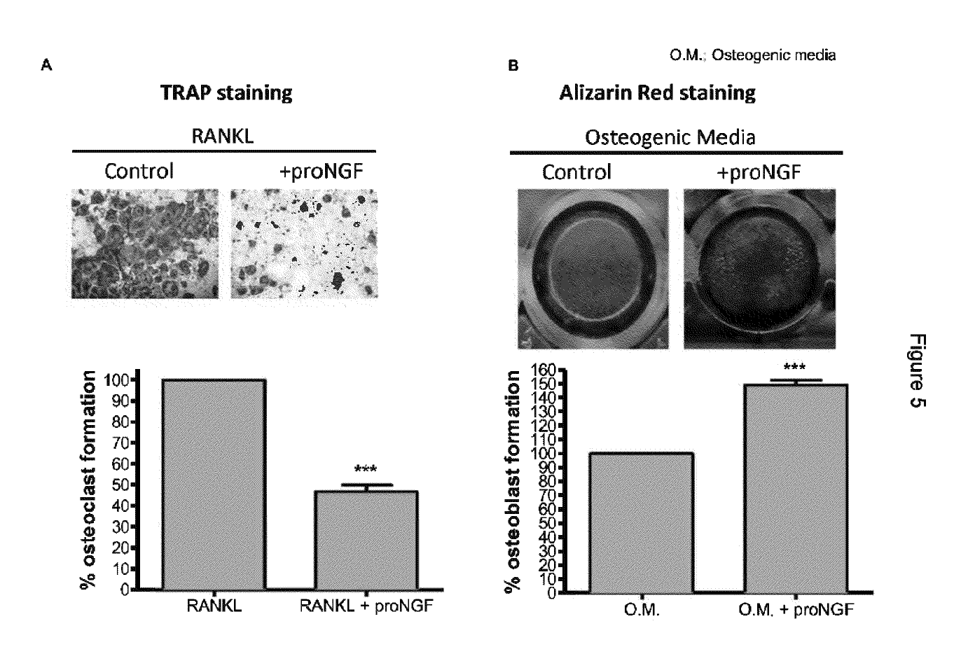

FIG. 6 demonstrates that recombinant NGF, BDNF, proNGF and proBDNF modulate osteoclast and osteoblast differentiation in vitro and mimics the phenotype seen in transgenic mice. A) WT mice (n=5 each) osteoclast primary culture cells were fixed and stained for TRAP after being cultured for 7 days in the presence of M-CSF/RANKL alone or with recombinant NGF (10 ng/ml), proNGF (20 ng/ml), BDNF (10 ng/ml) and proBDNF (10 ng/ml). TRAP-positive cells containing three or more nuclei were counted as osteoclasts. B) WT mice (n=5 each) osteoblast primary culture cells were fixed and stained for Alizarin Red after being cultured for 10 days in the presence of osteogenic media (αMEM containing 1 μM dexamethasone, 50 μg/ml ascorbic acid, and 10 mM β-glycerophosphate). Alizarin Red staining intensity was quantified using SigmaScan software. All data are expressed as mean±SEM. ***p<0.001 *p<0.05 vs. control (ANOVA).

FIG. 7 demonstrates that p75, sortilin, SorCS2 and TrkB receptors are expressed during osteoclast differentiation. p75, sortilin, SorCS2 and TrKB expression were analyzed in hematopoietic precursors (precursors), and daily up to 7 days after RANKL stimulation (MCSF1 and 2 and RANKL1 to 7) in WT mice (n=5). All data is expressed as mean±SEM. A) p75 fold change. B) Sortilin fold change. C) SorCS2 fold change. D) TrkB fold change.

FIG. 8 demonstrates that p75, Sortilin, SorCS2 and TrkB receptors are expressed during osteoclast differentiation and exert a role in differentiation. WT mice (n=5 each) osteoclast primary culture cells were fixed and stained for TRAP after being cultured for 7 days in the presence of M-CSF/RANKL alone or with recombinant NGF (10 ng/ml), proNGF (20 ng/ml), BDNF (10 ng/ml) and proBDNF (10 ng/ml) in combination with p75, Sortilin and SorCS2 antibodies. The histochemical stains of each combination are shown in A). TRAP-positive cells containing three or more nuclei were counted as osteoclasts. The percent of osteoclast differentiation from each combination is represent graphically in B). All data are expressed as means±SEM. ***p<0.001 vs. control (ANOVA).

FIG. 9 demonstrates that p75, Sortilin and Sorcs2 receptors are expressed during osteoblast differentiation. p75, sortilin and SorCS2 expression were analyzed in mesenchymal precursors (precursors), and daily up to 10 days in the presence of osteogenic media (αMEM containing 1 μM dexamethasone, 50 μg/ml ascorbic acid, and 10 mM β-glycerophosphate) in WT mice (n=5). A) demonstrates the observed p75 fold change. B) demonstrates the observed Sortilin fold change. C) demonstrates the observed Sorcs2 fold change. All data are expressed as mean±SEM.

FIG. 10 demonstrates that proNGF and proBDNF act via p75 to inhibit osteoclast differentiation. WT and p75−/− mice (n=3 each) osteoclast primary culture cells were fixed and stained for TRAP after being cultured for 7 days in the presence of M-CSF/RANKL alone or with recombinant NGF (10 ng/ml), proNGF (20 ng/ml), BDNF (10 ng/ml) and proBDNF (long/ml). The histochemical stains of each combination are shown in A). TRAP-positive cells containing three or more nuclei were counted as osteoclasts. The percent of TRAP-positive cells from each combination in wild type (WT) and p75 knockout mice is represent graphically in B). All data is expressed as mean±SEM. ***p<0.001 vs. control (ANOVA).

FIG. 11 demonstrates that proNGF and proBDNF act via p75 to promote osteoblast differentiation. WT and p75−/− mice (n=3 each) osteoblast primary culture cells were fixed and stained for Alizarin Red after being cultured for 10 days in the presence of osteogenic media (αMEM containing 1 μM dexamethasone, 50 μg/ml ascorbic acid, and 10 mM β-glycerophosphate) alone or with recombinant NGF (10 ng/ml), proNGF (20 ng/ml), BDNF (10 ng/ml) and proBDNF (long/ml). Cells were fixed and Alizarin Red staining intensity was quantified using SigmaScan software. The histochemical stains of each combination are shown in A). The Alizarin red expressed as a percent of control from each combination in wild type (WT) and p75 knockout mice is represent graphically in B). All data are expressed as mean±SEM. ***p<0.001 vs. control (ANOVA).

### DETAILED DESCRIPTION OF THE INVENTION

Before the present methods and treatment methodologies are described, it is to be understood that this invention is not limited to particular methods and experimental conditions described, as such methods and conditions may vary. It is also to be understood that the terminology used herein is for purposes of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth in their entirety.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference I their entireties.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

### DEFINITIONS

The terms used herein have the meanings recognized and known to those of skill in the art, however, for convenience and completeness, particular terms and their meanings are set forth below.

“Agent” refers to all materials that may be used to prepare pharmaceutical and diagnostic compositions, or that may be compounds such as small synthetic or naturally derived organic compounds, nucleic acids, polypeptides, antibodies, fragments, isoforms, variants, or other materials that may be used independently for such purposes, all in accordance with the present invention.

By “agonist” is meant a substance that binds to a specific receptor and triggers a response in a cell. It mimics the action of an endogenous ligand (such as hormone or neurotransmitter) that binds to the same receptor. A “full agonist” binds (has affinity for) and activates a receptor, displaying full efficacy at that receptor. One example of a drug that acts as a full agonist is isoproterenol which mimics the action of acetylcholine at β adrenoreceptors. A “partial agonist” (such as buspirone, aripiprazole, buprenorphine, or norclozapine) also binds and activates a given receptor, but has only partial efficacy at the receptor relative to a full agonist. A “partial agonist” may also be considered a ligand that displays both agonistic and antagonistic effects—when both a full agonist and partial agonist are present, the partial agonist actually acts as a competitive antagonist, competing with the full agonist for receptor occupancy and producing a net decrease in the receptor activation observed with the full agonist alone. A “co-agonist” works with other co-agonists to produce the desired effect together. An antagonist blocks a receptor from activation by agonists. Receptors can be activated or inactivated either by endogenous (such as hormones and neurotransmitters) or exogenous (such as drugs) agonists and antagonists, resulting in stimulating or inhibiting a biological response. A ligand can concurrently behave as agonist and antagonist at the same receptor, depending on effector pathways.

The potency of an agonist is usually defined by its EC₅₀ value. This can be calculated for a given agonist by determining the concentration of agonist needed to elicit half of the maximum biological response of the agonist. Elucidating an EC₅₀ value is useful for comparing the potency of drugs with similar efficacies producing physiologically similar effects. The lower the EC₅₀, the greater the potency of the agonist, and the lower the concentration of drug that is required to elicit a maximum biological response.

“Antagonist” refers to an agent that down-regulates (e.g., suppresses or inhibits) at least one bioactivity of a protein. An “antagonist” or an agent that “antagonizes” may be a compound which inhibits or decreases the interaction between a protein and another molecule, e.g., a target peptide or enzyme substrate. An antagonist may also be a compound that down-regulates expression of a gene or which reduces the amount of expressed protein present. Methods for assessing the ability of an agent to “antagonize” or “inhibit” a receptor are known to those skilled in the art.

“Analog” or “homolog” as used herein, refers to a chemical compound, a nucleotide, a protein, or a polypeptide that possesses similar or identical activity or function(s) as the chemical compounds, nucleotides, proteins or polypeptides having the desired activity and therapeutic effect of the present invention (e.g. to treat or prevent bone disease, or to modulate osteoblast or osteoclast activation, differentiation or function), but need not necessarily comprise a compound that is similar or identical to those compounds of the preferred embodiment, or possess a structure that is similar or identical to the agents of the present invention. In some instances, an analog or homolog of a compound such as a protein or peptide may have about 75%, 80%, 85%, 90%, 95% or 99% or more sequence homology to the compound.

“Derivative” refers to the chemical modification of molecules, either synthetic organic molecules or proteins, nucleic acids, or any class of small molecules such as fatty acids, or other small molecules that are prepared either synthetically or isolated from a natural source, such as a plant, that retain at least one function of the active parent molecule, but may be structurally different. Chemical modifications may include, for example, replacement of hydrogen by an alkyl, acyl, or amino group. It may also refer to chemically similar compounds which have been chemically altered to increase bioavailability, absorption, or to decrease toxicity. A derivative polypeptide is one modified by glycosylation, pegylation, or any similar process that retains at least one biological or immunological function of the polypeptide from which it was derived.

As used herein, the term “sequence homology” in all its grammatical forms refers to the relationship between proteins that possess a “common evolutionary origin,” including proteins from superfamilies (e.g., the immunoglobulin superfamily) and homologous proteins from different species (e.g., myosin light chain, etc.) (Reeck et al., Cell 50:667 (1987)). \

Accordingly, the term “sequence similarity” in all its grammatical forms refers to the degree of identity or correspondence between nucleic acid or amino acid sequences of proteins that do not share a common evolutionary origin (See, Reeck et al., Cell 50:667 (1987)). However, in common usage and in the instant application, the term “homologous,” when modified with an adverb such as “substantially,” may refer to sequence similarity and not a common evolutionary origin.

In a specific embodiment, two amino acid sequences are “substantially homologous” or “substantially similar” when greater than 30% of the amino acids are identical, or greater than about 60% are similar (functionally identical). Preferably, the similar or homologous sequences are identified by alignment using, for example, the GCG (Genetics Computer Group, Program Manual for the GCG Package, Version 7, Madison, Wis.) pileup program with the default parameters. Two amino acid sequences are also “substantially homologous” or “homologous” or “substantially similar” when greater than 75%, 80%, 85%, 90%, 95% or 99% or more of the amino acids are identical, or greater than about 75%, 80%, 85%, 90%, 95% or 99% or more of the amino acids are similar (functionally identical).

A “fragment” of a protein, peptide or amino acid sequence may be, for instance, about 30%, 50%, 60%, 75%, 80%, 85%, 90%, or 95% or more as long as the naturally occurring full length sequence of the protein, peptide or amino acid sequence. A “fragment” may possess all of, more than, or about 30%, 50%, 60%, 75%, 80%, 85%, 90%, or 95% or more of the biological activity of the naturally occurring full length sequence of the protein, peptide or amino acid sequence.

Mutations can be made in the sequences encoding the protein or peptide sequences of the proteins, peptides or polypeptides of the invention, such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein.

The following is one example of various groupings of amino acids:

Amino Acids with Nonpolar R Groups

### Alanine, Valine, Leucine, Isoleucine, Proline, Phenylalanine, Tryptophan, Methionine

Amino Acids with Uncharged Polar R Groups

### Glycine, Serine, Threonine, Cysteine, Tyrosine, Asparagine, Glutamine

Amino Acids with Charged Polar R Groups (Negatively Charged at Ph 6.0)
Aspartic acid, Glutamic acid

### Basic Amino Acids (Positively Charged at pH 6.0)

### Lysine, Arginine, Histidine (at pH 6.0)

Another Grouping May be Those Amino Acids with Phenyl Groups:

### Phenylalanine, Tryptophan, Tyrosine

Another grouping may be according to molecular weight (i.e., size of R groups):

| | | | |
|---|---|---|---|
| Glycine | 75 | Alanine | 89 |
| Serine | 105 | Proline | 115 |
| Valine | 117 | Threonine | 119 |
| Cysteine | 121 | Leucine | 131 |
| Isoleucine | 131 | Asparagine | 132 |
| Aspartic acid | 133 | Glutamine | 146 |
| Lysine | 146 | Glutamic acid | 147 |
| Methionine | 149 | Histidine (at pH 6.0) | 155 |
| Phenylalanine | 165 | Arginine | 174 |
| Tyrosine | 181 | Tryptophan | 204 |

Particularly preferred substitutions are:
Lys for Arg and vice versa such that a positive charge may be maintained;
Glu for Asp and vice versa such that a negative charge may be maintained;
Ser for Thr such that a free —OH can be maintained; and
Gln for Asn such that a free NH₂ can be maintained.

Exemplary and preferred conservative amino acid substitutions include any of: glutamine (Q) for glutamic acid (E) and vice versa; leucine (L) for valine (V) and vice versa; serine (S) for threonine (T) and vice versa; isoleucine (I) for valine (V) and vice versa; lysine (K) for glutamine (Q) and vice versa; isoleucine (I) for methionine (M) and vice versa; serine (S) for asparagine (N) and vice versa; leucine (L) for methionine (M) and vice versa; lysine (L) for glutamic acid (E) and vice versa; alanine (A) for serine (S) and vice versa; tyrosine (Y) for phenylalanine (F) and vice versa; glutamic acid (E) for aspartic acid (D) and vice versa; leucine (L) for isoleucine (I) and vice versa; lysine (K) for arginine (R) and vice versa.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

A “small molecule” refers to a molecule that has a molecular weight of less than 3 kilodaltons (kDa), preferably less than about 1.5 kilodaltons, more preferably less than about 1 kilodalton. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify compounds that modulate a bioactivity. A “small organic molecule” is normally an organic compound (or organic compound complexed with an inorganic compound (e.g., metal)) that has a molecular weight of less than 3 kilodaltons, and preferably less than 1.5 kilodaltons, and more preferably less than about 1 kDa.

“Diagnosis” or “screening” refers to diagnosis, prognosis, monitoring, characterizing, selecting patients, including participants in clinical trials, and identifying patients at risk for or having a particular disorder or clinical event or those most likely to respond to a particular therapeutic treatment, or for assessing or monitoring a patient's response to a particular therapeutic treatment.

The concept of “combination therapy” is well exploited in current medical practice. Treatment of a pathology by combining two or more agents that target the same pathogen or biochemical pathway sometimes results in greater efficacy and diminished side effects relative to the use of the therapeutically relevant dose of each agent alone. In some cases, the efficacy of the drug combination is additive (the efficacy of the combination is approximately equal to the sum of the effects of each drug alone), but in other cases the effect can be synergistic (the efficacy of the combination is greater than the sum of the effects of each drug given alone). As used herein, the term “combination therapy” means the two compounds can be delivered in a simultaneous manner, e.g. concurrently, or one of the compounds may be administered first, followed by the second agent, e.g. sequentially. The desired result can be either a subjective relief of one or more symptoms or an objectively identifiable improvement in the recipient of the dosage.

“Differentiate” or “differentiation” as used herein, generally refers to the process by which precursor or progenitor cells differentiate into specific cell types. In the present invention, the term refers to the process by which pre-osteoblasts become osteoblasts or pre-osteoclasts become osteoclasts. Differentiated cells can be identified by their patterns of gene expression and cell surface protein expression. As used herein, the term “differentiate” refers to having a different character or function from the original type of tissues or cells. Thus, “differentiation” is the process or act of differentiating. The term “Osteoclast Differentiation” refers to the process whereby osteoclast precursors in the bone marrow become functional osteoclasts, and the term “Osteoblast Differentiation” refers to the process whereby osteoblast precursors in the bone marrow become functional osteoblasts.

“Modulation” or “modulates” or “modulating” refers to up regulation (i.e., activation or stimulation), down regulation (i.e., inhibition or suppression) of a response, or the two in combination or apart. As used herein, the term “modulating” as related to osteoclast differentiation, refers to the ability of a compound or agent to exert an effect on precursors to osteoclasts, or to alter the expression of at least one gene related to osteoclastogenesis. For example, expression of the following genes is modulated during osteoclastogenesis: DC-Stamp, tartrate resistant alkaline phosphatase (TRAP), cathepsin K, calcitonin receptor, and integrin.

As used herein, the term “candidate compound” or “test compound” or “agent” or “test agent” refers to any compound or molecule that is to be tested. As used herein, the terms, which are used interchangeably, refer to biological or chemical compounds such as simple or complex organic or inorganic molecules, peptides, proteins, oligonucleotides, polynucleotides, carbohydrates, or lipoproteins. A vast array of compounds can be synthesized, for example oligomers, such as oligopeptides and oligonucleotides, and synthetic organic compounds based on various core structures, and these are also included in the terms noted above. In addition, various natural sources can provide compounds for screening, such as plant or animal extracts, and the like. Compounds can be tested singly or in combination with one another. Agents or candidate compounds can be randomly selected or rationally selected or designed. As used herein, an agent or candidate compound is said to be “randomly selected” when the agent is chosen randomly without considering the specific interaction between the agent and the target compound or site. As used herein, an agent is said to be “rationally selected or designed”, when the agent is chosen on a nonrandom basis which takes into account the specific interaction between the agent and the target site and/or the conformation in connection with the agent's action.

“Treatment” or “treating” refers to therapy, prevention and prophylaxis and particularly refers to administering medicine or performing medical procedures on a patient, for either prophylaxis (prevention) or to cure or reduce the extent of or likelihood of occurrence of the infirmity or malady or condition or event. In the present invention, the treatments using the agents described may be provided to stimulate or promote bone regeneration, to slow or halt bone loss, or to increase the amount or quality of bone density. Most preferably, the treating is for the purpose of stimulating or promoting bone regeneration or reducing or diminishing bone resorption. Treating as used herein also means administering the compounds for increasing bone density or for modulating osteoblastogenesis or osteoclastogenesis in individuals. Furthermore, in treating a subject, the compounds of the invention may be administered to a subject already suffering from loss of bone mass or other bone disease as provided herein or to prevent or inhibit the occurrence of such condition.

“Subject” or “patient” refers to a mammal, preferably a human, in need of treatment for a condition, disorder or disease.

“Osteoclastogenesis” refers to osteoclast generation, which is a multi-step process that can be reproduced in vitro. Earlier in vitro osteoclastogenesis systems used mixtures of stromal or osteoblastic cells together with osteoclast precursors from bone marrow (Suda, et al., (1997) Methods Enzymol. 282, 223-235; David et al., (1998) J Bone Miner. Res. 13, 1730-1738). These systems utilized 1α, 25-dihydroxyvitamin D₃ to stimulate stromal/osteoblastic cells to produce factors that support osteoclast formation More recent models utilize bone marrow cells cultured with soluble forms of the cytokines M-CSF (macrophage-colony stimulating factor) and a soluble form of RANKL (receptor activator of nuclear factor KB ligand) (Lacey, et al., (1998) Cell 93, 165-176; Shevde et al., (2000) Proc. Natl. Acad. Sci. U.S.A. 97, 7829-7834). These two cytokines are now recognized as the major factors from stromal cells that support osteoclastogenesis (Takahashi, et al., (1999) Biochem. Biophys. Res. Commun. 256, 449-455). Thus, their addition to the culture medium overcomes the need for stromal cells.

“Osteoclast precursor” refers to a cell or cell structure, such as a pre-osteoclast, which is any cellular entity on the pathway of differentiation between a macrophage and a differentiated and functional osteoclast. The term osteoclast includes any osteoclast-like cell or cell structure which has differentiated fully or partially from a macrophage, and which has osteoclast character, including but not limited to positive staining for tartrate-resistant acid phosphatase (TRAP), but which is not a fully differentiated or functional osteoclast, including particularly aberrantly differentiated or non functional osteoclasts or pre-osteoclasts.

“Osteoclast culture” refers to any in vitro or ex vivo culture or system for the growth, differentiation and/or functional assessment of osteoclasts or osteoclast precursors, whether in the absence or presence of other cells or cell types, for instance, but not limited to, osteoblasts, macrophages, hematopoietic or stromal cells.

“Osteoclast function,” as used herein, refers to bone resorption and the processes required for bone resorption.

An “amount sufficient to inhibit osteoclast differentiation, activation or function” refers to the amount sufficient to block either the differentiation, the formation or the function of osteoclasts, more particularly, an amount ranging from about 0.1 nM to about 10 μM, or more preferentially from about 0.1 nM to about 5 μM, and most preferentially from about 0.1 nM to about 1 μM in vitro. In vivo amounts of an agent sufficient to block either the differentiation, the formation or the function of osteoclasts may range from about 0.1 mg/Kg of body weight per day to about 200 mg/Kg of body weight per day in vivo, or more preferentially from about 1 mg/Kg to about 100 mg/Kg, and most preferentially from about 25 mg/Kg to about 50 mg/Kg of body weight per day in vivo. It is understood that the dose, when administered in vivo, may vary depending on the clinical circumstances, such as route of administration, age, weight and clinical status of the subject in which inhibition of osteoclast differentiation, formation or function is desired.

An “amount sufficient to stimulate or increase osteoblast differentiation, activation or function” refers to the amount of an agent sufficient to increase or stimulate either the differentiation, the activation or the function of osteoblasts, more particularly, an amount ranging from about 0.1 nM to about 10 μM, or more preferentially from about 0.1 nM to about 5 μM, and most preferentially from about 0.1 nM to about 1 μM in vitro. In vivo amounts of an agent sufficient to stimulate or increase either the differentiation, the activation or the function of osteoblasts may range from about 0.1 mg/Kg of body weight per day to about 200 mg/Kg of body weight per day in vivo, or more preferentially from about 1 mg/Kg to about 100 mg/Kg, and most preferentially from about 25 mg/Kg to about 50 mg/Kg of body weight per day in vivo. It is understood that the dose, when administered in vivo, may vary depending on the clinical circumstances, such as route of administration, age, weight and clinical status of the subject in which stimulation of increase of osteoblast differentiation, activation or function is desired.

In a specific embodiment, the term “about” means within 20%, preferably within 10%, and more preferably within 5% or even within 1%.

An “effective amount” or a “therapeutically effective amount” is an amount sufficient to stimulate or promote bone regeneration or decrease or prevent the symptoms associated with the conditions disclosed herein, including bone loss or in a decrease in bone mass or density, such as that which occurs with medical prosthetic devices or other related conditions contemplated for therapy with the compositions of the present invention. For example, an “effective amount” for therapeutic uses is the amount of the composition comprising an active compound herein required to provide reversal or inhibition of bone loss. Such effective amounts may be determined using routine optimization techniques and are dependent on the particular condition to be treated, the condition of the subject, the route of administration, the formulation, and the judgment of the practitioner and other factors evident to those skilled in the art. The dosage required for the compounds of the invention is that which induces a statistically significant difference in bone mass between treatment and control groups. This difference in bone mass or bone loss may be seen, for example, as at least 1-2%, or any clinically significant increase in bone mass or reduction in bone loss in the treatment group. The “effective amount” or “therapeutically effective amount” may range from about 1 mg/Kg to about 200 mg/Kg in vivo, or more preferentially from about 10 mg/Kg to about 100 mg/Kg, and most preferentially from about 25 mg/Kg to about 50 mg/Kg in vivo.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

Binding compounds can also be characterized by their effect on the activity of the target molecule. Thus, a “low activity” compound has an inhibitory concentration (IC₅₀) (for inhibitors or antagonists) or effective concentration (EC₅₀) (applicable to agonists) of greater than 1 μM under standard conditions. By “very low activity” is meant an IC₅₀ or EC₅₀ of above 100 μM under standard conditions. By “extremely low activity” is meant an IC₅₀ or EC₅₀ of above 1 mM under standard conditions. By “moderate activity” is meant an IC₅₀ or EC₅₀ of 200 nM to 1 μM under standard conditions. By “moderately high activity” is meant an IC₅₀ or EC₅₀ of 1 nM to 200 nM. By “high activity” is meant an IC₅₀ or EC₅₀ of below 1 nM under standard conditions. The IC₅₀ (or EC₅₀) is defined as the concentration of compound at which 50% of the activity of the target molecule (e.g., enzyme or other protein) activity being measured is lost (or gained) relative to activity when no compound is present. Activity can be measured using methods known to those of ordinary skill in the art, e.g., by measuring any detectable product or signal produced by occurrence of an enzymatic reaction, or other activity by a protein being measured.

An individual “at risk” may or may not have detectable disease, and may or may not have displayed detectable disease prior to the treatment methods described herein. “At risk” denotes that an individual who is determined to be more likely to develop a symptom based on conventional risk assessment methods or has one or more risk factors that correlate with development of a bone disease or low bone mass or density or enhanced susceptibility to bone resorption. An individual having one or more of these risk factors has a higher probability of developing bone resporption than an individual without these risk factors.

“Prophylactic” or “therapeutic” treatment refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

Antibodies, including polyclonal and monoclonal antibodies, particularly antibodies to receptors for pro-neurotrophins or to pro-neurotrophins themselves and neutralizing antibodies may be useful as compounds to modulate osteoclast differentiation and/or function. These antibodies are available from such vendors as Upstate Biologicals, Santa Cruz, or they made be prepared using standard procedures for preparation of polyclonal or monoclonal antibodies known to those skilled in the art. Also, antibodies including both polyclonal and monoclonal antibodies, and drugs that modulate the activity of the adenosine receptor and/or its subunits may possess certain diagnostic applications and may for example, be utilized for the purpose of detecting and/or measuring conditions such as bone diseases, bone loss, or osteoclast differentiation and/or function. The pro-neurotrophin receptors or their subunits may be used to produce both polyclonal and monoclonal antibodies to themselves in a variety of cellular media, by known techniques such as the hybridoma technique utilizing, for example, fused mouse spleen lymphocytes and myeloma cells. Likewise, small molecules that mimic or act as agonists for the activities of the pro-neurotrophin receptor may be discovered or synthesized, and may be used in diagnostic and/or therapeutic protocols.

Antibodies to Nerve Growth Factor (NGF) are undergoing testing for the treatment of osteoarthritis and other chronic painful conditions. An uncommon but surprising toxicity of this treatment is osteonecrosis, commonly resulting in joint replacement. NGF is secreted as a pro-peptide that is converted extracellularly to NGF by proteolysis of the furin site in the protein.

Recent studies indicate that proNGF is an active modulator of bone metabolism. Mice that make large quantities of a mutated form of proNGF that cannot be cleaved into NGF were examined. These mice had significantly thicker (and stronger) bones than mice that did not make excess proNGF that could not be cleaved. In early experiments cultured mouse osteoblasts were treated with proNGF. The osteoblasts made more bone in culture. Interestingly, when osteoclasts were treated with proNGF, osteoclast formation in culture was suppressed. These findings are consistent with the observation that mice with excess proNGF have increased bone density and suggest that proNGF is involved in maintaining bone strength and structure, and absence of proNGF may lead to bone and joint pathology.

The present invention provides, in part, a mutated, poorly hydrolyzed form of proNGF that both stimulates osteoblast differentiation and inhibits osteoclast differentiation. When studied in vivo in transgenic mice that overexpress the mutated, poorly hydrolysable proNGF produces a marked increase in bone density, consistent with the in vitro findings, NGF binds to its receptor TrkA whereas proNGF is a signaling molecule that binds to p75NTR, sortilin and SorCS2. Similarly, mice transgenic for a gene that overexpresses proBDNF have the same phenotype. Prior work has demonstrated that p75NTR and sortilin are expressed on osteoblastic cell lines and periodontal cells where, when stimulated, they upregulate osteoblast differentiation (Maeda, et al., Journal of Cellular Physiology, 2002; 193(1): 73-9; Mikami, et al., Differentiation Research in Biological Diversity, 2012; 84(5): 392-9; Kurihara, et al., Journal of Periodontology, 2003; 74(1): 76-84), although this signaling was induced by BDNF or NGF, not the pro-peptides,

The present invention provides mutated proNGF or proBDNF peptides that are poorly hydrolyzed to treat diseases of bone including, for instance, avascular necrosis, neuropathic arthropathy (Charcot Joint), osteoporosis, metastatic lesions, etc. In addition the present invention provides pro NGF or pro BDNF to promote bone growth, regeneration and fracture healing. Unlike other currently available therapies for osteoporosis that target only the osteoclast this approach targets both osteoblasts and osteoclasts.

These agents may be administered intravenously, subcutaneously or even orally. Alternatively, small molecule agonists at these receptors could be used to treat bone diseases, as noted above.

### Role of Neurotrophins and Pro-Neurotrophins in Bone Metabolism

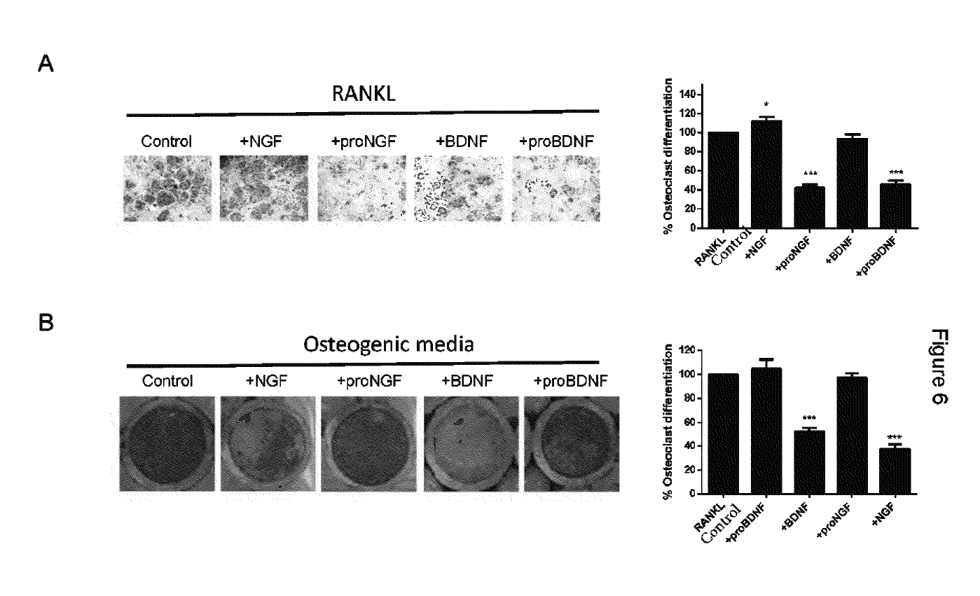

As shown in FIG. 6, NGF stimulates a modest increase in osteoclast differentiation (FIG. 6A) whereas both proNGF and proBDNF markedly inhibit osteoclast differentiation. Some data regarding osteoblast differentiation demonstrate that both BDNF and NGF inhibit osteoblast differentiation, and some data demonstrate no detectable effect of either proNGF or proBDNF on osteoblast differentiation. The absence of an effect of the pro-neurotrophins on osteoblast differentiation according to these particular data may be due to the manner in which the data were collected so that at the termination of the experiments the cells had already maximally differentiated and it was not possible to detect enhancement.

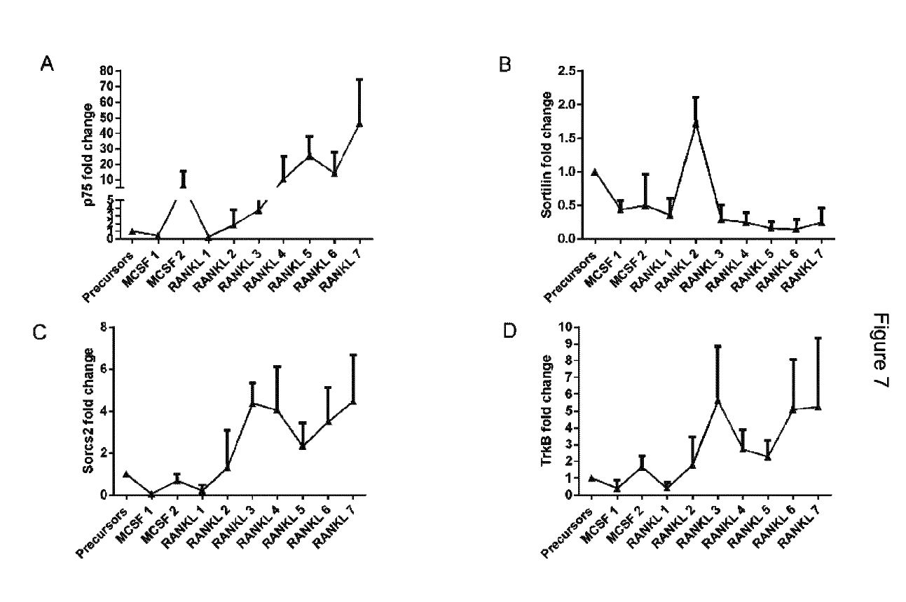

As shown in FIG. 7, p75 increases throughout osteoclast differentiation reaching a peak in fully mature osteoclasts. In contrast, sortilin is most highly expressed after initial exposure to RANKL. Both SorCS2 and TrkB are increasingly expressed throughout osteoclast differentiation.

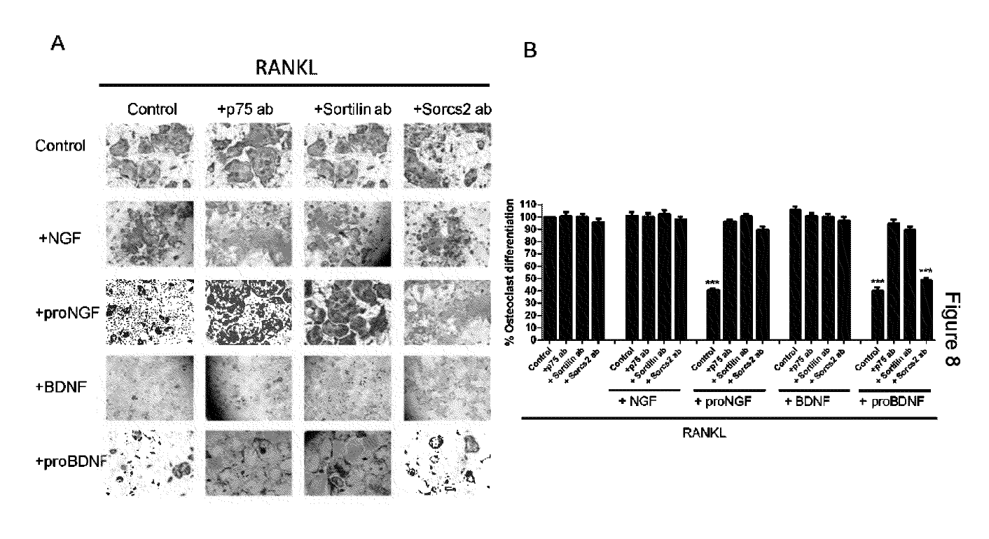

Antibodies to p75, sortilin and SorCS2 are available and useful to demonstrate the capacity of proNGF or proBDNF to inhibit osteoclast differentiation. This capacity was examined. As shown in FIG. 8, anti-p75, anti-sortilin and anti-SorCS2 reverse the effects of proNGF on osteoclast differentiation whereas anti-p75 and anti-sortilin but not anti-SorCS2 antibodies reversed the effect of proBDNF on osteoclast differentiation.

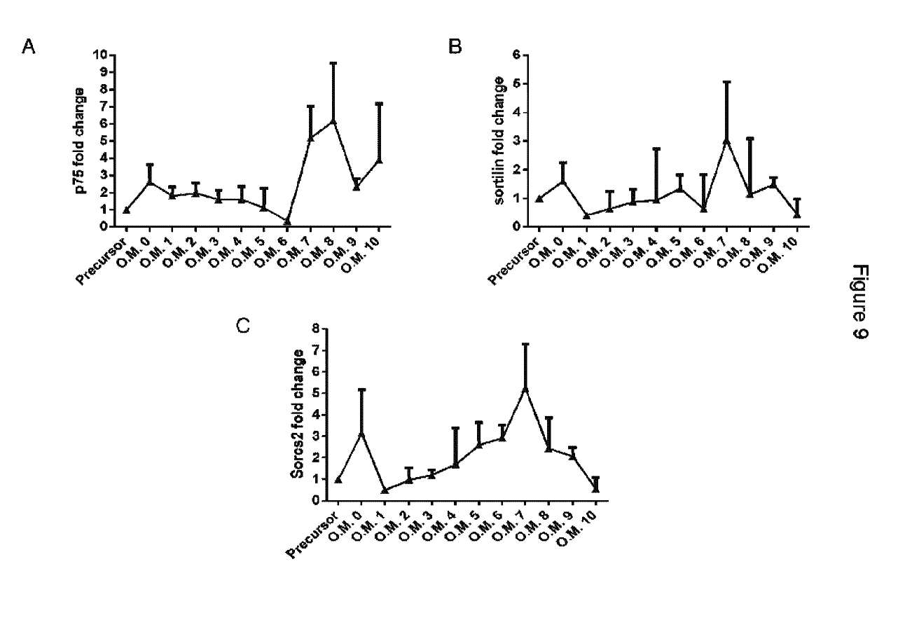

As with osteoclast differentiation, osteoblasts expressed increasing quantities of mRNA for all 3 receptors during differentiation with some variation in the kinetics of the increase (FIG. 9). This further indicate that pro-neurotrophins regulate osteoclast and osteoblast differentiation via specific receptors for pro-neurotrophins, as previously described in mice with gain of function and in earlier studies.

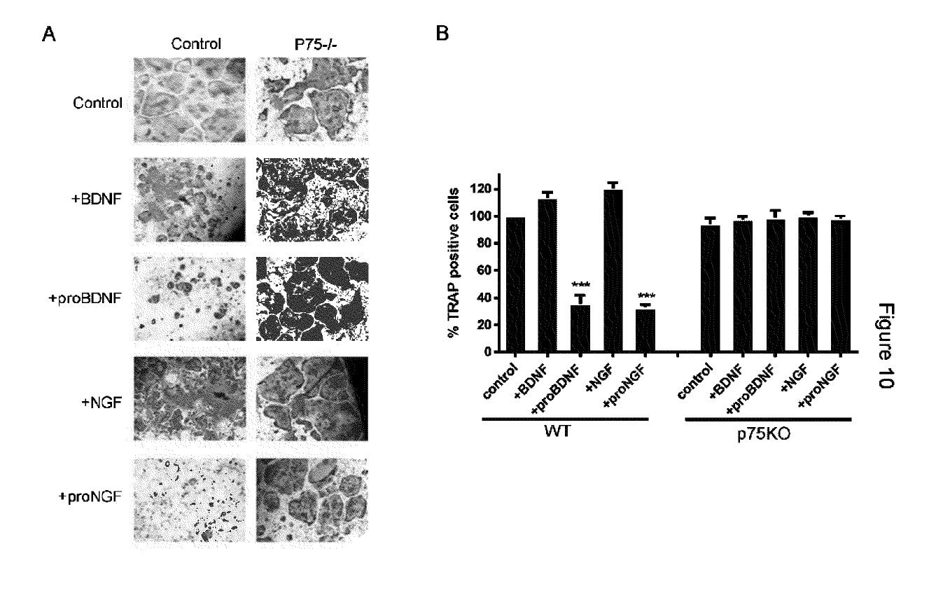

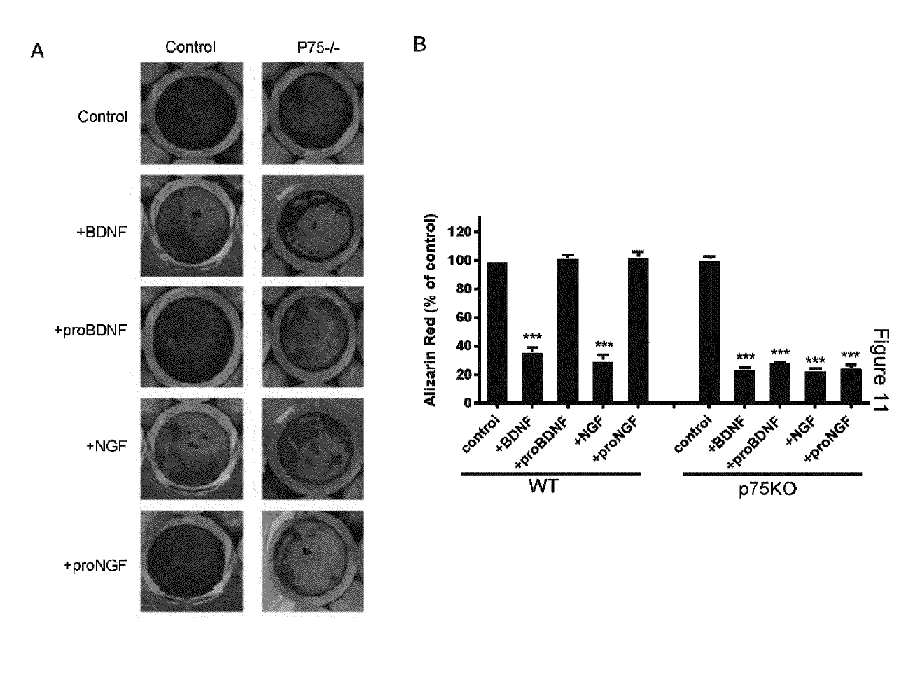

The effect of proBDNF, BDNF, proNGF and NGF on osteoclast and osteoblast differentiation was examined using bone marrow obtained from animals lacking p75 (p75−/−), which have a normal bone phenotype. As shown in FIG. 10, in the absence of p75, neither proNGF nor proBDNF inhibited osteoclast differentiation. In contrast, both BDNF and NGF inhibited osteoblast differentiation in cells from wild type animals and neither proBDNF nor proNGF altered osteoblast differentiation, as noted above (FIG. 11). In contrast, in the absence of p75 there was a significant reduction in osteoblast differentiation in p75 KO cells exposed to either proBDNF and proNGF. These findings demonstrate that osteoblast differentiation depends on endogenous ligation and activation of p75 for osteoblast differentiation and that excess stimulation of neurotrophin receptors inhibits osteoblast differentiation.

Therapeutic and Prophylactic Compositions and their Use

Candidates for therapy with the agents and methods described herein are patients suffering from bone diseases, procedures involving bone injury, or bone resorption.

The invention provides methods of treatment featuring administering to a subject an effective amount of an agent of the invention. The compound is preferably substantially purified (e.g., substantially free from substances that limit its effect or produce undesired side-effects). The subject is preferably an animal, including but not limited to animals such as monkeys, cows, pigs, horses, chickens, cats, dogs, etc., and is preferably a mammal, and most preferably human. In one specific embodiment, a non-human mammal is the subject. In another specific embodiment, a human mammal is the subject. Accordingly, the agents identified by the methods described herein may be formulated as pharmaceutical compositions to be used for prophylaxis or therapeutic use to treat these patients.

Various delivery systems are known and can be used to administer a compound of the invention, e.g., encapsulation in liposomes, microparticles, or microcapsules. Methods of introduction can be enteral or parenteral and include but are not limited to intradermal, intramuscular, intraperitoneal, intravenous, subcutaneous, intranasal, epidural, topical and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. In a specific embodiment, it may be desirable to administer the pharmaceutical compositions of the invention locally to the area in need of treatment.

Such compositions comprise a therapeutically effective amount of an agent, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In another embodiment, the compound can be delivered in a vesicle, in particular a liposome (Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327)

In yet another embodiment, the compound can be delivered in a controlled or sustained release system. In one embodiment, a pump may be used (see Langer, supra; Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials can be used (See, Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger et al., (1983) Macromol. Sci. Rev. Macromol. Chem. 23:61; Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, i.e., the subject bone or prosthesis, thus requiring only a fraction of the systemic dose (see, e.g., Goodson, in Medical Applications of Controlled Release (1984) supra, vol. 2, pp. 115-138). Other suitable controlled release systems are discussed in the review by Langer (1990) Science 249:1527-1533.

The present invention further contemplates therapeutic compositions useful in practicing the therapeutic methods of this invention. A subject therapeutic composition may include, in admixture, a pharmaceutically acceptable excipient (carrier) and one or more of a proNGF or proBDNF or an analog, homolog, fragment or derivative thereof, such as, for instance, a poorly hydrolyzed fragment thereof.

Effects of the compounds or agents of the invention can first be tested for their ability to stimulate or mimic using standard techniques known in the art. More particularly, the selectivity of the compounds for the receptor can be assessed using radioligand binding assays whereby a test or candidate compound can be assayed for its ability to bind to a cell having or expressing the receptor. Cells can be transfected with the nucleic acid encoding the various adenosine receptors and competitive binding assays with radiolabeled ligands run to evaluate the specificity of the particular candidate compounds.

The compounds or compositions of the invention may be combined for administration with or embedded in polymeric carrier(s), biodegradable or biomimetic matrices or in a scaffold. The carrier, matrix or scaffold may be of any material that will allow composition to be incorporated and expressed and will be compatible with the addition of cells or in the presence of cells. Preferably, the carrier matrix or scaffold is predominantly non-immunogenic and is biodegradable. Examples of biodegradable materials include, but are not limited to, polyglycolic acid (PGA), polylactic acid (PLA), hyaluronic acid, catgut suture material, gelatin, cellulose, nitrocellulose, collagen, albumin, fibrin, alginate, cotton, or other naturally-occurring biodegradable materials. It may be preferable to sterilize the matrix or scaffold material prior to administration or implantation, e.g., by treating it with ethylene oxide or by gamma irradiation or irradiation with an electron beam. In addition, a number of other materials may be used to form the scaffold or framework structure, including but not limited to: nylon (polyamides), dacron (polyesters), polystyrene, polypropylene, polyacrylates, polyvinyl compounds (e.g., polyvinylchloride), polycarbonate (PVC), polytetrafluorethylene (PTFE, teflon), thermanox (TPX), polymers of hydroxy acids such as polylactic acid (PLA), polyglycolic acid (PGA), and polylactic acid-glycolic acid (PLGA), polyorthoesters, polyanhydrides, polyphosphazenes, and a variety of polyhydroxyalkanoates, and combinations thereof. Matrices suitable include a polymeric mesh or sponge and a polymeric hydrogel. In the preferred embodiment, the matrix is biodegradable over a time period of less than a year, more preferably less than six months, most preferably over two to ten weeks. The polymer composition, as well as method of manufacture, can be used to determine the rate of degradation. For example, mixing increasing amounts of polylactic acid with polyglycolic acid decreases the degradation time. Meshes of polyglycolic acid that can be used can be obtained commercially, for instance, from surgical supply companies (e.g., Ethicon, N.J.). A hydrogel is defined as a substance formed when an organic polymer (natural or synthetic) is cross-linked via covalent, ionic, or hydrogen bonds to create a three-dimensional open-lattice structure which entraps water molecules to form a gel. In general, these polymers are at least partially soluble in aqueous solutions, such as water, buffered salt solutions, or aqueous alcohol solutions, that have charged side groups, or a monovalent ionic salt thereof.

For use in treating animal subjects, the compositions of the invention can be formulated as pharmaceutical or veterinary compositions. Depending on the subject to be treated, the mode of administration, and the type of treatment desired, e.g., prevention, prophylaxis, therapy; the compositions are formulated in ways consonant with these parameters. A summary of such techniques is found in Remington's Pharmaceutical Sciences, latest edition, Mack Publishing Co., Easton, Pa.

The preparation of therapeutic compositions containing small organic molecules polypeptides, analogs or active fragments as active ingredients is well understood in the art. The compositions of the present invention may be administered parenterally, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Formulations may be prepared in a manner suitable for systemic administration or for topical or local administration. Systemic formulations include, but are not limited to those designed for injection (e.g., intramuscular, intravenous or subcutaneous injection) or may be prepared for transdermal, transmucosal, nasal, or oral administration. Such compositions may be prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. The formulation will generally include a diluent as well as, in some cases, adjuvants, buffers, preservatives and the like. In addition, if desired, the composition can contain minor amounts of auxiliary substances such as wetting or emulsifying agents, pH buffering agents which enhance the effectiveness of the active ingredient.

A small organic molecule/compound, a polypeptide, an analog or active fragment thereof can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like. For oral administration, the compositions can be administered also in liposomal compositions or as microemulsions. Suitable forms include syrups, capsules, tablets, as is understood in the art.

The compositions of the present invention may also be administered locally to sites in subjects using a variety of techniques known to those skilled in the art. For example, these may include sprays, lotions, gels or other vehicles such as alcohols, polyglycols, esters, oils and silicones. The administration of the compositions of the present invention may be pharmacokinetically and pharmacodynamically controlled by calibrating various parameters of administration, including the frequency, dosage, duration mode and route of administration. Variations in the dosage, duration and mode of administration may also be manipulated to produce the activity required. The therapeutic compositions are conventionally administered in the form of a unit dose, for instance intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the agent selected for treating the subject, the dosage formulation, and in a therapeutically effective amount. If one desires to achieve the desired effect in vitro, the effective amounts may range from about 0.1 nM to about 10 μM, more preferably about 0.1 nM to about 5 μM, and most preferably from about 0.1 nM to about 1 nM. The desired effect refers to the effect of the agent on reducing or inhibiting osteoclast differentiation or stimulation, reducing or inhibiting bone resorption and stimulating or increasing osteoblast differentiation, activation or function. Precise amounts of active ingredient required to be administered depend on the judgment of the practitioner and are peculiar to each individual. However, suitable dosages to achieve the desired therapeutic effect in vivo may range from about 0.1 mg/kg body weight per day to about 200 mg/kg body weight per day, or from about 1.0 mg/kg body weight per day to about 100 mg/kg body weight per day, preferably about 25 mg/kg body weight per day to about 50 mg/kg body weight per day. In a particular embodiment, the term “about” means within 20%, preferably within 10%, and more preferably within 5%. The preferred dose will depend on the route of administration. However, dosage levels are highly dependent on the nature of the disease or situation, the condition of the subject, the judgment of the practitioner, and the frequency and mode of administration. If the oral route is employed, the absorption of the substance will be a factor effecting bioavailability. A low absorption will have the effect that in the gastro-intestinal tract higher concentrations, and thus higher dosages, will be necessary. Suitable regimes for initial administration and further administration are also variable, but are typified by an initial administration followed by repeated doses at one or more hour intervals by a subsequent injection or other administration. Alternatively, continuous intravenous infusion sufficient to maintain desired concentrations, e.g. in the blood, are contemplated. The composition may be administered as a single dose multiple doses or over an established period of time in an infusion.

It will be understood that the appropriate dosage of the substance should suitably be assessed by performing animal model tests, where the effective dose level (e.g., ED₅₀) and the toxic dose level (e.g. TD₅₀) as well as the lethal dose level (e.g. LD₅₀ or LD₁₀) are established in suitable and acceptable animal models. Further, if a substance has proven efficient in such animal tests, controlled clinical trials should be performed.

The compounds or compositions of the present invention may be modified or formulated for administration at the site of pathology. Such modification may include, for instance, formulations which facilitate or prolong the half-life of the compound or composition, particularly in the environment. Additionally, such modification may include the formulation of a compound or composition to include a targeting protein or sequence which facilitates or enhances the uptake of the compound/composition to bone or bone precursor cells. In a particular embodiment, such modification results in the preferential targeting of the compound to bone or bone precursor cells versus other locations or cells. In one embodiment, a tetracycline, tetracycline family or bisphosphonate may be utilized to target the compound or composition of the present invention to bone or bone cells, including osteoclasts and osteoclast precursors. Novel heterocycles as bone targeting compounds are disclosed in U.S. Patent Publication No. 2002/0103161 A₁, which is incorporated herein by reference in its entirety.

Pharmaceutically acceptable carriers useful in these pharmaceutical compositions include, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Sterile injectable forms of the compositions may be aqueous or oleaginous suspensions. The suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Parenteral formulations may be a single bolus dose, an infusion or a loading bolus dose followed with a maintenance dose. These compositions may be administered once a day or on an “as needed” basis. The pharmaceutical compositions may be orally administered in any orally acceptable dosage form including, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. Alternatively, the pharmaceutical compositions may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols. The pharmaceutical compositions of this invention may also be administered topically. Topical application can be effected in a rectal suppository formulation (see above) or in a suitable enema formulation. Topically-transdermal patches may also be used. For topical applications, the pharmaceutical compositions may be formulated in a suitable ointment containing the active component suspended or dissolved in one or more carriers. Carriers for topical administration of the compounds of this invention include, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum. The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The invention also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### Effective Doses

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects can be used, care should be taken to design a delivery system that targets such compounds to the site of affected tissue in order to minimize potential damage to unaffected cells and, thereby, reduce side effects.

The data obtained from cell culture assays and animal studies can be used in formulating a dose range for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to optimize efficacious doses for administration to humans. Plasma levels can be measured by any technique known in the art, for example, by high performance liquid chromatography.

In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. Normal dose ranges used for particular therapeutic agents employed for specific diseases can be found in the Physicians' Desk Reference 54^{th} Edition (2000).

### EXAMPLES

The following examples are set forth to provide those of ordinary skill in the art with a description of how to make and use the methods and compositions of the invention, and are not intended to limit the scope thereof. Efforts have been made to insure accuracy of numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Osteoclast Differentiation

Wild type C57BL/6, proNGF/+, proBDNF/+, BDNF+/− and TrkB+/− mice were sacrificed in a CO₂ chamber and the marrow cavity was flushed out with α-MEM from femora and tibiae aseptically removed, and bone marrow was incubated overnight in α-MEM containing 10% FBS and 1% penicillin/streptomycin (αMEM) to obtain a single-cell suspension. 200,000 non-adherent cells were collected and seeded in α-MEM with 30 ng/ml M-CSF for two days. At day 3 (day 0 of differentiation), 30 ng/ml RANKL was added to the culture. Cultures were fed every third day by replacing the culture medium with fresh medium and reagents. After incubation for 7 days, cells were prepared for tartrate-resistant acid phosphatase (TRAP) staining for osteoclast quantification (Mediero, et al. Sci Transl Med., 2012; 4: 135ra165; Mediero, et al., Am J Pathol., 2012; 180: 775-786). The number of TRAP-positive MNCs containing ≧3 nuclei/cell were scored (Yasuda, et al. Bone, 1999; 25: 109-113). To determine the effect of proNGF on osteoclast culture, recombinant proNGF was added to the culture at the time of RANKL.

### Osteoblast Differentiation

Mouse bone marrow-derived osteoblasts culture was performed as previously described. Briefly, BMCs were isolated by flushing out the bone marrow cavity. BMCs were cultured for 3 days, non-adherent cells were discarded, and the adherent cells were cultured until confluent. The stromal cells were then washed and reseeded in culture dishes at 1×10⁵ cell/cm² density with osteogenic medium (αMEM containing 1 μM dexamethasone, 50 ng/ml ascorbic acid, and 10 mM β-glycerophosphate). To test the osteogenesis, Alizarin Red staining was performed 10 days after culture. Cells were fixed in 4% PFA and stained for 45 minutes with 2% Alizarin Red. To determine the effect of proNGF on osteoclast culture, recombinant proNGF was added to the culture at the time of the osteogenic media

### Micro-X-Ray Computed Tomography (Micro-CT) Analysis of Bone Mass

For measurements of the bone volume (trabecular bone volume (BV/TV)), the femurs of wild type (WT) and C57BL/6, proNGF/+, proBDNF/+, BDNF+/− and TrkB+/− mice were measured by micro-CT, as previously described (Mediero, et al. Sci Transl Med., 2012; 4: 135ra165). Briefly, analyses were performed in the μCT core at the Hospital for Special Surgery in New York, N.Y. using the Scanco Medical MicroCT 35 Scanner with 25-nm resolution (KVp: 5T μA/45) and every field of view was scanned using a CCD detector, with an integration time of 400 ms. For qualitative analysis, 3D images of the femur were then reconstructed from the cross-sectional slices using the software provided by Scanco Medical MicroCT 35 and processing was done to get direct morphometric measurements in 3D.

### Western Blot

For western blot analysis primary mouse BMCs were treated with 30 ng/ml RANKL and 30 ng/ml M-CSF or osteogenic media for 24 hours (n=4 each). Cells were lysed with RIPA buffer containing protease/phosphatase inhibitors and centrifuged at 13,000 rpm to collect supernatant. Protein concentration was determined by BCA and μg of protein was subjected to SDS-PAGE and transferred to a nitrocellulose membrane. To block nonspecific binding, membranes were treated in TBS/Tween-20 0.05% with 5% skimmed milk 1 hour at room temperature, and membranes where incubated overnight 4° C. with primary antibodies against p75 1:250 and sortilin 1:1000. After washing with TBS/Tween-20 0.05% membranes were incubated with goat anti-rabbit IRDye 800CW 1:10000 and goat anti-mouse IRDye 680 RD 1:10000 (Li-cor Biosciences) one hour at room temperature in the dark. Proteins were visualized by Li-cor Odyssey equipment where near-infrared fluorescent signal were detected. As each secondary antibody emits a signal in a different spectrum, reprobing with actin (to check that all lanes were loaded with the same amount of protein) was performed at the same time as the primary antibody incubation. Intensities of the respective band are quantitated by densitometric analysis using the Image Studio 2.0.38 software Li-cor Biosciences.

To quantify Western blot analysis digital densitometric band analysis was performed and band intensities were expressed relative to actin. Variations in intensity expressed as % of control and expressed as mean±SEM. All results were calculated as a percentage of non-stimulated controls to minimize the intrinsic variation among different experiments. Statistical analysis was performed using a one-way ANOVA and Bonferroni post-hoc test and the levels of significance are indicated in the figure legends, provided above.

### Quantitative Real-Time RT-PCR

To confirm the expression of osteoclast differentiation markers, primary mouse BMCs cells were cultured for seven days in the presence of 30 ng/ml of RANKL and 30 ng/ml M-CSF. For the osteogenesis assays, primary mouse stromal cells were incubated for 10 days with osteogenic media (n=2 each assay). Expression levels were quantitated by quantitative RT-PCR (qRT-PCR). Cells were collected at established time points during the differentiation studies, and total RNA was extracted using RNeasy Mini Kit (Qiagen, Invitrogen), including sample homogenization with QIA shredder columns. Oncolumn DNA digestion was performed to avoid genomic DNA contamination. 0.5 μg of total RNA was retrotranscribed using MuLV Reverse transcriptase PCR kit (Applied Biosystems, Foster City, Calif., USA) at 2.5 U/μl, including in the same reaction Rnase Inhibitor 1 U/μl, Random Hexamers 2.5 U/μlm MgCl₂ 5 mM, PCR buffer II 1× and dNTPs 1 mM. Real-Time RT-PCR was used to relatively quantify gene expression using iQ SYBR green Supermix (Bio-Rad). The following primers were used: Cathepsin K Forward:

5′-GCTGAACTCAGGACCTCTGG-3′
and
Reverse:
5′-GAAAAGGGAGGCATGAATGA-3′;
NFATc1 Forward:
5′-TCATCCTGTCCAACACCAAA-3′
and
Reverse:
5′-TCACCCTGGTGTTCTTCCTC-3′;
Osteopontin Forward:
5′-TCTGATGAGACCGTCACTGC-3′
and
Reverse:
5′-TCTCCTGGCTCTCTTTGGAA-3;
RANKL Forward
5′-AGCCGAGACTACGGCAAGTA-3′
and
Reverse:
5′-GCGCTCGAAAGTACAGGAAC-3′;
OPG Forward
5′-GGCAACACAGCTCACAAGAA-3′
and
Reverse:
5′-CTGGGTTTGCATGCCTTTAT-3′
and
GAPDH Forward:
5′-CTACACTGAGGACCAGGTTGTCT-3′
and
Reverse:
5′-GGTCTGGGATGGAAATTGTG-3′.

The Pfaffl method (Pfaffl, Nucleic Acids Res., 2001; 29: e45) was used for relative quantification.

### Example 2

The effect of proNGF on bone metabolism will be further explored. This will allow intervening to prevent joint deformity in patients with peripheral neuropathy and to develop therapies for other types of bone problems that might share this mechanism (e.g. avascular necrosis of bone, a common problem in people taking high doses of cortisone-like agents among a number of other causes).

The effect of proNGF on bone metabolism will be examined in mice. The results of the initial experiments discussed above will be confirmed and expanded. The bones of transgenic mice that make excess poorly hydrolyzed proNGF will be examined and compared to the bones of normal mice using microCT examination of the bones. Bone marrow cells will be isolated from the transgenic mice and normal mice. The change in osteoclast or osteoblast (the cells involved in bone metabolism) differentiation and function will be determined. Using a variety of specific antibodies, the receptor(s) for proNGF on osteoclasts and osteoblasts will be identified.

The similar effect of proNGF on human bone cells will be confirmed. Similar experiments to those described above will be performed on human osteoclasts and osteoblasts. Human bone marrow is purchased from commercial suppliers for these experiments.

The intracellular signaling of bone cells in response to proNGF will be examined. The intracellular signals that mediate increased osteoblast and osteoclast function in human and mouse precursor cells will be examined. Cells isolated from mice and humans as well as appropriate cell lines representative of these cell types will be used.

Transgenic mice that overexpress proNGF may be protected from developing destructive bone changes in a mouse model of osteonecrosis. Because there are no accepted animal models of neuropathic arthropathy, the bone response of transgenic and normal mice will be compared in response to high dose corticosteroid (dexamethasone) in an animal model of steroid-induced osteonecrosis. Identifying the cellular receptors involved in proNGF-mediated stimulation of osteoblasts and suppression of osteoclasts will facilitate the process of finding a drug that has the appropriate effects on the receptor.

### Example 3

The role of proNGF and proBDNF in the regulation of bone metabolism was further examined. As shown in FIG. 6, NGF stimulates a modest increase in osteoclast differentiation (FIG. 6A) whereas both proNGF and proBDNF markedly inhibit osteoclast differentiation. In contrast, present data regarding osteoblast differentiation demonstrate that both BDNF and NGF inhibit osteoblast differentiation whereas there was no detectable effect of either proNGF or proBDNF on osteoblast differentiation. The absence of an effect of the pro-neurotrophins on osteoblast differentiation may be due to the manner in which the study was performed so that at the termination of the study, the cells had already maximally differentiated. As such, it was not possible to detect enhancement.

To determine the effect of differentiation into osteoclasts on expression of receptors for pro-neurotrophins or BDNF, the message expression for p75, SorCS2, sortilin and TrkB during osteoclast differentiation was examined. As shown in FIG. 7, p75 increases throughout osteoclast differentiation reaching a peak in fully mature osteoclasts. In contrast, sortilin is most highly expressed after initial exposure to RANKL. Both SorCS2 and TrkB are increasingly expressed throughout osteoclast differentiation.

To better understand which pro-neurotrophin and neurotrophin receptors play a role in regulation of osteoclast function by pro-neurotrophins, the effect of antibodies to p75, sortilin and SorCS2 on the capacity of proNGF or proBDNF to inhibit osteoclast differentiation were examined. As shown in FIG. 8, anti-p75, anti-sortilin and anti-SorCS2 reversed the effects of proNGF on osteoclast differentiation whereas anti-p75 and anti-sortilin but not anti-sorcs2 antibodies reversed the effect of proBDNF on osteoclast differentiation.

As with osteoclast differentiation, osteoblasts expressed increasing quantities of mRNA for all 3 receptors during differentiation with some variation in the kinetics of the increase (FIG. 9). These results further indicate that pro-neurotrophins regulate osteoclast and osteoblast differentiation via specific receptors for pro-neurotrophins, as previously described in mice with gain of function and in earlier studies.

To confirm the role of p75 in osteoclast and osteoblast differentiation bone marrow was obtained from animals lacking p75 (p75−/−), which have a normal bone phenotype. The effect of proBDNF, BDNF, proNGF and NGF on osteoclast and osteoblast differentiation was examined. As shown in FIG. 10, in the absence of p75, neither proNGF nor proBDNF inhibited osteoclast differentiation. In contrast, both BDNF and NGF inhibited osteoblast differentiation in cells from wild type animals and neither proBDNF nor proNGF altered osteoblast differentiation, as noted above (FIG. 11). In contrast, in the absence of p75 there was a significant reduction in osteoblast differentiation in p75 KO cells exposed to either proBDNF and proNGF. These findings demonstrate that osteoblast differentiation depends on endogenous ligation and activation of p75 for osteoblast differentiation and that excess stimulation of neurotrophin receptors inhibits osteoblast differentiation.

1. A method for stimulating or promoting bone growth, bone density or bone regeneration comprising administering to a subject a therapeutically effective amount of a pro-nerve growth factor (proNGF) or a pro-brain derived neurotrophic factor (proBDNF) or a biologically active analog, homolog, fragment or derivative thereof.

2. The method according to claim 1 wherein the pro-nerve growth factor (proNGF) or the pro-brain derived neurotrophic factor (proBDNF) is a mutated or a poorly hydrolyzed form of proNGF or proBDNF.

3. The method according to claim 1 wherein the pro-nerve growth factor (proNGF) or the pro-brain derived neurotrophic factor (proBDNF) stimulates osteoblast differentiation or activation or inhibits osteoclast differentiation or activation.

4. A method for stimulating differentiation or activation of osteoblasts or increasing function of osteoblasts comprising administering to a subject a therapeutically effective amount of a pro-nerve growth factor (proNGF) or a pro-brain derived neurotrophic factor (proBDNF) or a biologically active analog, homolog, fragment or derivative thereof.

5. The method according to claim 4 wherein the pro-nerve growth factor (proNGF) or the pro-brain derived neurotrophic factor (proBDNF) is a mutated or a poorly hydrolyzed form of proNGF or proBDNF.

6. The method according to claim 4 wherein the pro-nerve growth factor (proNGF) or the pro-brain derived neurotrophic factor (proBDNF) stimulates osteoblast differentiation or activation or inhibits osteoclast differentiation or activation.

7. A method for inhibiting differentiation or activation of osteoclasts or decreasing function of osteoclasts comprising administering to a subject a therapeutically effective amount of a pro-nerve growth factor (proNGF) or a pro-brain derived neurotrophic factor (proBDNF) or a biologically active analog, homolog, fragment or derivative thereof.

8. The method according to claim 7 wherein the pro-nerve growth factor (proNGF) or the pro-brain derived neurotrophic factor (proBDNF) is a mutated or a poorly hydrolyzed form of proNGF or proBDNF.

9. The method according to claim 7 wherein the pro-nerve growth factor (proNGF) or the pro-brain derived neurotrophic factor (proBDNF) stimulates osteoblast differentiation or activation or inhibits osteoclast differentiation or activation.

10. A method for treating, ameliorating or preventing a bone disease or a condition in a subject having such a bone disease or in a subject at risk for developing such a bone disease comprising administering to the subject a therapeutically effective amount of a pro-nerve growth factor (proNGF) or a pro-brain derived neurotrophic factor (proBDNF) or a biologically active analog, homolog, fragment or derivative thereof.

11. The method according to claim 10 wherein the pro-nerve growth factor (proNGF) or the pro-brain derived neurotrophic factor (proBDNF) is a mutated or a poorly hydrolyzed form of proNGF or proBDNF.

12. The method according to claim 10 wherein the pro-nerve growth factor (proNGF) or the pro-brain derived neurotrophic factor (proBDNF) stimulates osteoblast differentiation or activation or inhibits osteoclast differentiation or activation.

13. The method according to claim 10 wherein the bone disease or condition is selected from the group consisting of osteoporosis, juvenile osteoporosis, bone loss due to or associated with the onset of menopause, osteoporotic fractures, giant cell tumors of bone, renal osteodystrophy, osteogenesis imperfecta, hypercalcemia, hyperparathyroidism, osteomalacia, osteohalisteresis, osteolytic bone disease, osteonecrosis, Paget's disease of bone, bone loss due to rheumatoid arthritis, inflammatory arthritis, osteomyelitis, corticosteroid treatment, metastatic bone diseases or malignancy-induced osteoporosis and bone lysis, childhood idiopathic bone loss, periodontal bone loss, age-related loss of bone mass, osteotomy and bone loss associated with prosthetic ingrowth.

14. The method according to claim 10 wherein the bone disease is selected from the group consisting of avascular necrosis, neuropathic arthropathy (Charcot Joint), osteoporosis and metastatic lesions.

15. The method according to claim 10 wherein the bone disease is osteonecrosis.

16. A pharmaceutical composition comprising a pro-nerve growth factor (proNGF) or a biologically active analog, homolog, fragment or derivative thereof.

17. A pharmaceutical composition comprising a pro-brain derived neurotrophic factor (proBDNF) or a biologically active analog, homolog, fragment or derivative thereof.

18. A pharmaceutical composition according to claim 16 further comprising one or more compounds or agents effective for inhibiting bone resorption, for inhibiting osteoclast differentiation or activation or for promoting osteoblast differentiation or activation.

19. A pharmaceutical composition according to claim 17 further comprising one or more compounds or agents effective for inhibiting bone resorption, for inhibiting osteoclast differentiation or activation or for promoting osteoblast differentiation or activation.


SEQUENCE LISTING (12 sequences)

SEQ ID NO: 1 - DNA, 20 bp, Artificial Sequence
  Synthetic oligonucleotide
gctgaactcaggacctctgg

SEQ ID NO: 2 - DNA, 20 bp, Artificial Sequence
  Synthetic oligonucleotide
gaaaagggaggcatgaatga

SEQ ID NO: 3 - DNA, 20 bp, Artificial Sequence
  Synthetic oligonucleotide
tcatcctgtccaacaccaaa

SEQ ID NO: 4 - DNA, 20 bp, Artificial Sequence
  Synthetic oligonucleotide
tcatcctgtccaacaccaaa

SEQ ID NO: 5 - DNA, 20 bp, Artificial Sequence
  Synthetic oligonucleotide
tctgatgagaccgtcactgc

SEQ ID NO: 6 - DNA, 20 bp, Artificial Sequence
  Synthetic oligonucleotide
tctcctggctctctttggaa

SEQ ID NO: 7 - DNA, 20 bp, Artificial Sequence
  Synthetic oligonucleotide
agccgagactacggcaagta

SEQ ID NO: 8 - DNA, 20 bp, Artificial Sequence
  Synthetic oligonucleotide
gcgctcgaaagtacaggaac

SEQ ID NO: 9 - DNA, 20 bp, Artificial Sequence
  Synthetic oligonucleotide
ggcaacacagctcacaagaa

SEQ ID NO: 10 - DNA, 20 bp, Artificial Sequence
  Synthetic oligonucleotide
ctgggtttgcatgcctttat

SEQ ID NO: 11 - DNA, 23 bp, Artificial Sequence
  Synthetic oligonucleotide
ctacactgaggaccaggttgtct

SEQ ID NO: 12 - DNA, 20 bp, Artificial Sequence
  Synthetic oligonucleotide
ggtctgggatggaaattgtg